(19) United States
(12) Patent Application Publication
(10) Pub. No.: US 2025/0391576 A1
(43) Pub. Date: Dec. 25, 2025

(54) METHODS FOR PREDICTING AND SELECTING NEOANTIGENS

(71) Applicant: Natera, Inc, San Carlos, CA (US)

(72) Inventors: Robert BURNS, Brookfield, WI (US); Helio COSTA, Jackson, WY (US); Matthew RABINOWITZ, Sunny Isles Beach, FL (US); Miller RICHTERS, University, MO (US); Jasreet HUNDAL, St. Louis, MO (US); Alexey ALESHIN, San Francisco, CA (US)

(21) Appl. No.: 19/242,354

(22) Filed: Jun. 18, 2025

Related U.S. Application Data
(60) Provisional application No. 63/661,737, filed on Jun. 19, 2024.

(51) Int. Cl. G16H 50/50; G16B 15/30; G16B 30/00; G16H 20/10

(52) U.S. Cl. CPC G16H 50/50; G16B 15/30; G16B 30/00; G16H 20/10

(57) ABSTRACT

A system and corresponding method are provided for identifying a subpopulation of cancer patients who are immunotherapy respondents. An effective method of assessing neoantigen presentation is provided. A vaccine composition is also provided. The vaccine composition is prepared by feeding data for a subject with a type of cancer into a predictive model and scoring neoantigens that occur in data for the subject for one or more parameters. One or more vaccine compositions to be administered to the subject are prepared for one or more somatic mutations for one or more neoantigens that satisfy an immune stimulation threshold.

This patent application claims benefit of U.S. Provisional Patent Application Ser. No. 63/661,737, filed on Jun. 19, 2024, which is incorporated by reference in its entirety for all purposes.

### BACKGROUND

Tumor mutational burden (TMB) that reflects the number of cancer mutations has emerged as a predictive biomarker of immunotherapy. Although high TMB status leads to increased neoantigen presentation and enables T-cell recognition, not all mutations produce neoantigens, or elicit an immune response, which makes TMB an imperfect biomarker. Accordingly, a need exists for an improved method for identifying a subpopulation of cancer patients who are immunotherapy respondents using a more effective method of assessing neoantigen presentation. Such a method will also enable the creation of more effective therapies based on presented neoantigens.

### SUMMARY

In one aspect, the present disclosure relates to a method for identifying a cancer patient as an immunotherapy responder, comprising: performing whole exome sequencing and whole genome sequencing on a tumor sample of the patient to quantify the number of neoantigens in the tumor sample; performing RNA sequencing, such as whole transcriptome RNA sequencing or targeted T and B cell receptor sequencing using extracted DNA, on a tumor sample of the patient to quantify the number of unique T- and B-cell receptors and enrichment of immune cell populations in the tumor sample; identifying a cancer patient as an immunotherapy responder using the number of neoantigens in the tumor sample, the number of unique T- and B-cell receptors, the abundance of each unique T and B cell receptors, and the enrichment of immune cell populations in the tumor sample. In some examples, customized panels of about 100 to 500 cancer genes are used.

In some embodiments, the method further comprises performing whole exome sequencing on a germline sample and or the tumor sample of the patient to genotype MHC I and MHC Il alleles of the patient.

In some embodiments, quantifying neoantigens in the tumor sample comprises (i) genotyping MHC I and MHC II alleles of the patient by germline and or tumor whole exome sequencing; (ii) identifying somatic mutations in the tumor sample of the patient that cause changes in protein sequences and filtering out somatic mutations from unexpressed genes according to RNA sequencing of the tumor sample; and (iii) pairing each of the MHC I and MHC Il alleles of the patient obtained in (if) with each peptide of 8-12 (MHC I) or 10-30 (MHC II) amino acids in length that comprises at least one somatic mutation obtained in (ii), and identifying one or more neoantigens based on MHC-peptide binding and T-cell activation.

In some embodiments, the somatic mutations comprise single nucleotide variants (SNV), multi-nucleotide variants (MNVs), copy number variants (CNVs), indels, gene fusions, structural variants, or a combination thereof.

In some embodiments, the neoantigens are identified using one or more neoantigen classifiers.

In some embodiments, quantifying the number and abundance of unique T- and B-cell receptors comprises: (i) deconvoluting proportions of immune cells in the tumor sample based on RNA sequencing data, and (ii) assembling B and T-cell receptors to quantify the number of unique T- and B-cell receptors.

In some embodiments, enrichment of immune cell populations in the tumor sample is determined by tumor gene expression based on RNA sequencing data.

In some embodiments, the tumor sample of the patient is from a solid tumor.

In some embodiments, the method identifies a cancer patient as an immunotherapy responder with a positive predictive value (PPV) that is at least 10%, at least 20%, at least 30%, at least 40%, or at least 50% higher than use of TMB alone. In some embodiments, the method identifies a cancer patient as an immunotherapy non-responder with a negative predictive value (NPV) that is at least 10%, at least 20%, at least 30%, at least 40%, or at least 50% higher than use of TMB alone.

In some embodiments, the method identifies a cancer patient as an immunotherapy responder with a sensitivity that is at least 10%, at least 20%, at least 30%, at least 40%, or at least 50% higher than use of TMB alone. In some embodiments, the method identifies a cancer patient as an immunotherapy non-responder with a specificity that is at least 10%, at least 20%, at least 30%, at least 40%, or at least 50% higher than use of TMB alone.

In another aspect, the present disclosure relates to a method for treating cancer, comprising administering treatment to a cancer patient who has been identified as an immunotherapy responder by the method described herein.

In some embodiments, the treatment comprises a checkpoint inhibitor, a CAR-T therapy, a TCR-T therapy, a NK cell therapy, a cancer vaccines, an oncolytic virus, a cytokine, a monoclonal antibody, or a combination thereof.

In some embodiments, the immunotherapy comprises a PD1 inhibitor, a PD-L1 inhibitor, a CTLA-4 inhibitor, or a combination thereof.

In some embodiments, the immunotherapy comprises Pembrolizumab, Nivolumab, Cemiplimab, Dostarlimab, Atezolizumab, Avelumab, Durvalumab, Ipilimumab, or Tremelimumab. In some embodiments, the immunotherapy comprises Vopratelimab, Spartalizumab, Camrelizumab, Sintilimab, Tislelizumab, Toripalimab, INCMGA00012, AMP-224, AMP-514, KN035, Cosibelimab, AUNP12, CA-170, or BMS-986189.

In some embodiments, the cancer patient has been or is concurrently treated with surgery, chemotherapy, or radiation therapy.

In some embodiments, the cancer is breast cancer, colorectal cancer, gastrointestinal cancer, kidney cancer, lung cancer, bladder cancer, ovarian cancer, or pancreatic cancer.

In some embodiments, the cancer is a cancer or tumor of abdomen or abdominal wall, adrenal gland, anus, appendix, bladder, bone, brain, breast, cervix, chest wall, colon, diaphragm, duodenum, ear, endometrium, esophagus, fallopian tube, gallbladder, gastro-esophageal junction, head and neck, kidney, larynx, liver, lung, lymph node, malignant effusions, mediastinum, nasal cavity, omentum, ovarian, pancreas, pancreatobiliary, parotid gland, pelvis, penis, pericardium, peritoneum, pleura, prostate, rectum, salivary gland, skin, small intestine, soft tissue, spleen, stomach, thyroid, tongue, trachea, ureter, uterus, vagina, vulva, or whipple resection.

In a further aspect, the present disclosure relates to a method identifying a cancer patient as a PD-1 or PD-L1 or CTLA-4 immunotherapy responder, comprising: performing whole exome sequencing on a tumor sample of the patient to quantify the number of neoantigens in the tumor sample; performing RNA sequencing on a tumor sample of the patient to quantify the number of unique T- and B-cell receptors, enrichment of immune cell populations in the tumor sample, and expression of PD-1 or PD-L1 or CTLA-4; and identifying a cancer patient as an PD-1 or PD-L1 or CTLA-4 immunotherapy responder using the number of neoantigens in the tumor sample, the number and abundance of unique T- and B-cell receptors, the enrichment of immune cell populations in the tumor sample, and the expression of PD-1 or PD-L1 or CTLA-4.

In a further aspect, the present disclosure relates to a method for treating cancer, comprising administering an immunotherapy to a cancer patient who has been identified as a PD-1 or PD-L1 or CTLA-4 immunotherapy responder by the method described herein, wherein the immunotherapy comprises Pembrolizumab, Nivolumab, Cemiplimab, Dostarlimab, Atezolizumab, Avelumab, Durvalumab, Ipilimumab, or Tremelimumab.

In other embodiments, a method for generating a pool of about 10-100 personal cancer vaccines is provided. The personal cancer vaccines are matched to patients. Prediction models are developed using clinical genomics data. Clusters of patients with a type of cancer are identified. In examples, these clusters tend to have one or more neoantigens in common. In some examples, the prediction models utilize standard clustering algorithms. In other examples, the phylogenetic evolution or clonal evolution of the tumor is utilized to identify clusters of patients with a type of cancer.

Vaccines are built for each cluster of patients. In some examples, the vaccines cover the most immunogenic neoantigens in the centroid of each cluster. Patients, such as new patients with a type of cancer, may then be matched to vaccines that have built by identifying to which cluster the patient belongs and/or predicting an immune response score of the patient to each available vaccine. at least one computing device comprising at least one processor configured to:

In some embodiments, a system, method, non-transitory computer-readable medium for outputting a catalog of one or more somatic mutations is provided. Data from subjects is fed into a predictive model and neoantigens that occur in the data from a subset of the subjects are scored for one or more parameters. Based on scores for the one or more parameters, one or more neoantigens that occur in a subset of the subjects that satisfy an immune stimulation threshold are determined. Somatic mutations for the one or more neoantigens that satisfy the immune stimulation threshold that occur in the subset of the subjects are determined. A predicted catalog of one or more of the somatic mutations that occur the subset of the subjects that satisfy the immune stimulation threshold predict the somatic mutations is output.

In some aspects, data is from subjects previously treated for the type of cancer and the predictive model is a machine learning model. In other aspects, the predictive model is more predictive of immune stimulation for the subset of subjects than tumor mutational burden (TMB) status. The parameters may further comprise one or more of immune checkpoint inhibitor (ICI), response, ctDNA results, age, sex, and ECOG score.

In some embodiments, a system, method, non-transitory computer-readable medium for outputting one or more vaccines to be administered to a subject. Data from subjects is fed into a trained model. Neoantigens that occur in data for the subject are scored for one or more parameters. Based on scores for the one or more parameters, one or more neoantigens that occur for the subject that satisfy an immune stimulation threshold are determined. Somatic mutations for the one or more neoantigens that satisfy the immune stimulation threshold that occur for the subject are determined. One or more vaccines to be administered to the subject are output for the somatic mutations for the one or more neoantigens that satisfy the immune stimulation threshold. The one or more vaccines to be administered to the subject are selected from a predicted catalog of somatic mutations that occur in a subset of subjects previously treated for the type of cancer.

In aspects, the one or more vaccines are selected from a pool of pre-made vaccines to be administered. In other aspects, the vaccine is one or more of a peptide-based synthetic vaccine, messenger RNA (mRNA) vaccines, or traditional vaccine.

In other embodiments, a vaccine composition is provided. The vaccine composition is, prepared by a process comprising the steps of: feeding data for a subject with a type of cancer into a predictive model; scoring neoantigens that occur in data for the subject for one or more parameters; determining, based on scores for the one or more parameters, one or more neoantigens that occur for the subject, that satisfy an immune stimulation threshold; determining somatic mutations for the one or more neoantigens that satisfy the immune stimulation threshold that occur for the subject; and preparing one or more vaccine compositions to be administered to the subject for one or more somatic mutations for one or more neoantigens that satisfy an immune stimulation threshold where the one or more vaccines to be administered to the subject are selected from a predicted catalog of somatic mutations that occur in a subset of subjects previously treated for the type of cancer.

In aspects, the one or more parameters comprise a combination of one or more of peptide processing and presentation, RNA expression, MHC binding fold change, T-cell activation, and dissimilarity from reference human proteome. In other aspects, the one or more vaccine compositions are selected from a pool of pre-made vaccine compositions to be administered. The one or more vaccine compositions may be one or more of a peptide-based synthetic vaccine, messenger RNA (mRNA) vaccines, or traditional vaccine. In other aspects, the one or more neoantigens and liquid nanoparticle (LNP) are combined to prepare the one or more vaccine compositions.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1A is a block diagram of clinical data abstraction in accordance with examples set forth herein.

FIG. 1B is a block diagram of an exemplary immune response prediction model in accordance with examples set forth herein.

FIGS. 2A-2D are graphical representations of immune response for individuals with Melanoma in accordance with examples set forth herein.

FIGS. 3A-3D are graphical representations of immune response for individuals with NSCLC in accordance with examples set forth herein.

FIGS. 4A-4D are graphical representations of immune response for individuals with MSI-H CRC in accordance with examples set forth herein.

FIGS. 5A-5D are graphical representations of immune response for individuals with solid tumors in accordance with examples set forth herein.

FIG. 6 is a block diagram illustrating a computing system in accordance with examples set forth herein.

FIG. 7 is a block diagram illustrating a prediction engine in accordance with examples set forth herein.

FIG. 8 is a block diagram of a bioinformatics pipeline in accordance with examples set forth herein.

FIG. 9 is a flow diagram for a method for training immune stimulation prediction model in accordance with examples set forth herein.

FIG. 10 is a flow diagram of a method for generating an immune stimulation and vaccine prediction in accordance with examples set forth herein.

FIG. 11 is a block diagram of an exemplary bioinformatics pipeline in accordance with examples set forth herein.

FIG. 12 is a graphical representation of neoantigen prioritization performance in accordance with examples set forth herein.

FIG. 13 is a graphical representation of true-positive neoantigen prediction in accordance with examples set forth herein.

FIG. 14 is a graphical representation of the neoantigen prediction and prioritization method compared with other methods in accordance with examples set forth herein.

FIG. 15 is a graphical representation of RNAseq data used with the neoantigen prediction and prioritization method in accordance with examples set forth herein.

FIG. 16 is a graphical representation of the neoantigen prediction and prioritization method compared with TMB in accordance with examples set forth herein.

FIG. 17 is a graphical representation of ICI response and the neoantigen prediction and prioritization method in accordance with examples set forth herein.

### DETAILED DESCRIPTION

### General Overview

Methods and compositions provided herein improve immunotherapy for treatment of cancer. In one aspect, the present disclosure relates to a method for identifying a cancer patient as an immunotherapy responder, comprising: performing whole exome sequencing, whole genome sequencing, or customized panels on a tumor sample of the patient to quantify the number of neoantigens in the tumor sample; performing RNA sequencing on a tumor sample of the patient to quantify the number of unique T- and B-cell receptors and enrichment of immune cell populations in the tumor sample; identifying a cancer patient as an immunotherapy responder using the number of neoantigens in the tumor sample, the number and abundance of unique T- and B-cell receptors, and the enrichment of immune cell populations in the tumor sample.

In another aspect, the present disclosure relates to a method for treating cancer, comprising administering an immunotherapy to a cancer patient who has been identified as an immunotherapy responder by the method described herein.

In a further aspect, the present disclosure relates to a method identifying a cancer patient as a PD-1 or PD-L1 or CTLA-4 immunotherapy responder, comprising: performing whole exome sequencing, whole genome sequencing, or customized panels of cancer genes on a tumor sample of the patient to quantify the number of neoantigens in the tumor sample; performing RNA sequencing on a tumor sample of the patient to quantify the number of unique T- and B-cell receptors, enrichment of immune cell populations in the tumor sample, and expression of PD-1 or PD-L1 or CTLA-4; and identifying a cancer patient as an PD-1 or PD-L1 or CTLA-4 immunotherapy responder using the number of neoantigens in the tumor sample, the number and abundance of unique T- and B-cell receptors, the enrichment of immune cell populations in the tumor sample, and the expression of PD-1 or PD-L1 or CTLA-4.

In a further aspect, the present disclosure relates to a method for treating cancer, comprising administering an immunotherapy to a cancer patient who has been identified as a PD-1 or PD-L1 or CTLA-4 immunotherapy responder by the method described herein, wherein the immunotherapy comprises Pembrolizumab, Nivolumab, Cemiplimab, Dostarlimab, Atezolizumab, Avelumab, Durvalumab, Ipilimumab, or Tremelimumab.

### Samples Collection

The methods disclosed herein improves immunotherapy for treatment of a wide variety of cancers in a patient. A person of ordinary skill in the art would understand that different types of cancer will require collection of different type of samples as described herein.

In some embodiments, the cancer is a solid tumor, and the biological sample is a tumor biopsy sample. Performing a biopsy generally involves using a sharp tool to remove a small amount of tissue from a patient suspected to containing diseased cells or tissue such as a tumor. There are many different types of biopsies such as needle biopsy, CT-guided biopsy, ultrasound guided biopsy, bone biopsy, bone marrow biopsy, liver biopsy, kidney biopsy, aspiration biopsy, prostate biopsy, skin biopsy, surgical biopsy such as laparoscopic biopsy. In some embodiments, the biological sample is obtained by liquid biopsy. In some embodiments, the biological sample is a blood, serum, plasma, or urine sample. Further, biological liquid samples may be extracted from variety of animal fluids containing cell free DNA, including but not limited to blood, serum, plasma, bone marrow, urine vitreous, sputum, tears, perspiration, saliva, semen, mucosal excretions, mucus, spinal fluid, amniotic fluid, lymph fluid and so on. Cell free DNA may be fetal in origin (via fluid taken from a pregnant subject) or may be derived from tissue of the subject itself.

In some embodiments, the cancer is a blood cancer, and the biological sample is a liquid sample. In some embodiments, the cancer is a blood cancer, and the biological sample is blood, serum, plasma, or bone marrow sample. In some embodiments, the DNA from the cancer and the matched normal DNA are both obtained from the blood sample by isolating and separating plasma and buffy coat. The DNA obtained from the buffy coat may serve as the matched normal DNA to the circulating tumor DNA obtained from the plasma fraction.

In some embodiments, the methods of the present disclosure further comprise longitudinally collecting a plurality of liquid biopsy samples from the patient. In some embodiments, the liquid biopsy sample is obtained from the patient after the patient has been treated for the cancer. In some embodiments, the liquid biopsy sample is a blood, serum, plasma, or urine sample.

### Methods for Identifying Immunotherapy Respondents

The present disclosure relates to a method for identifying a cancer patient as an immunotherapy responder, comprising: performing whole exome sequencing, whole genome sequencing, or customized panel sequencing on a tumor sample of the patient to quantify the number of neoantigens in the tumor sample; performing RNA sequencing on a tumor sample of the patient to quantify the number and abundance of unique T- and B-cell receptors and enrichment of immune cell populations in the tumor sample; identifying a cancer patient as an immunotherapy responder using the number of neoantigens in the tumor sample, the number of unique T- and B-cell receptors, and the enrichment of immune cell populations in the tumor sample.

In some embodiments, the method further comprises performing whole exome sequencing on a germline sample of the patient to genotype MHC I and or MHC II alleles of the patient. In some embodiments, whole exome sequencing, whole genome sequencing, or customized panel sequencing is performed on cellular DNA obtained from a solid tumor and from matched normal tissue such as buffy coat. By comparing sequencing data of DNA obtained from the tumor sample with DNA obtained from normal matched tissue, neoantigens may be identified and used to determine whether a cancer patient is an immunotherapy responder.

In some embodiments, the term “whole exome sequencing” refers to sequencing of all protein coding regions of genes in a genome, also known as exomes. Whole exome sequencing of tumor biopsy samples is described in, for example, WO2015/164432 and WO2019/200228, which are incorporated by reference in their entireties.

In some embodiments, whole exome sequencing may first involve a step of isolating a subset of DNA encoding protein that are known as exons before sequencing. This first step may be performed by capture techniques to isolated exons, i.e., array based capture or in-solution capture as described elsewhere herein. Target-enrichment methods allow one to selectively capture genomic regions of interest from a DNA sample prior to sequencing by enrichment methods such as hybrid capture or targeted amplification. The genomic regions of interests may include all the exonic regions of the genome to prepare samples for whole exome sequencing (WES).

In some embodiments, quantifying neoantigens in the tumor sample comprises (i) genotyping MHC I and MHC II alleles of the patient by germline whole exome sequencing (or the other types of seq) or sequencing of the tumor sample; (ii) identifying somatic mutations in the tumor sample of the patient that cause changes in protein sequences and filtering out somatic mutations from unexpressed genes according to RNA sequencing of the tumor sample; and (iii) pairing each of the MHC I and MHC II alleles of the patient obtained in (i) with each peptide of 8-12 (MHC I) or 10-30 (MHC II) amino acids in length that comprises at least one somatic mutation obtained in (ii), and identifying one or more neoantigens based on MHC-peptide binding and T-cell activation.

In some embodiments, the somatic mutations identified in the tumor sample comprise single nucleotide variants (SNV), multi-nucleotide variants (MNVs), copy number variants (CNVs), indels, gene fusions, structural variants, aberrant splice variants, or a combination thereof. The term “indel” refers to both insertion and deletion of nucleic acids in the genome. The term “gene fusions” refers to any genomic alteration resulting in the fusion of two different genomic loci caused by insertions and/or deletions of DNA in the genome. The term “structural variant” refers to a genomic alteration such as deletions or insertions that involve DNA segments larger than 1 kilo base (kb) and could be either microscopic or submicroscopic.

In some embodiments, the somatic mutations identified in the tumor sample are protein-coding mutations. In some embodiments, the protein-coding mutations are of one or more oncogene, tumor suppressor genes, genes that enhance or inhibit cell proliferation, invasion, or metastasis, genes that promote or inhibit apoptosis, pro-angiogenesis or anti-angiogenesis genes. In some embodiments, the protein-coding mutations are of AKT1 (14q32.33, ALK (2p23.2-23.1), APC (5q22.2), AR (Xq12), ARAF (Xp11.3), ARID1A (1p36.11), ATM (11q22.3), BRAF (7q34), BRCA1 (17q21.31), BRCA2 (13q13.1), CCND1 (11q13.3), CCND2 (12p13.32), CCNE1 (19q12), CDH1 (16q22.1), CDK4 (12q14.1), CDK6 (7q21.2), CDKN2A (9p21.3), CTNNB1 (3p22.1), DDR2 (1q23.3), EGFR (7p11.2), ERBB2 (17q12), ESR1 (6q25.1-25.2), EZH2 (7q36.1), FBXW7 (4q31.3), FGFR1 (8p11.23), FGFR2 (10q26.13), FGFR3 (4p16.3), GATA3 (10p14), GNA11 (19p13.3), GNAQ (9q21.2), GNAS (20q13.32), HNF1A (12q24.31), HRAS (11p15.5), IDH1 (2q34), IDH2 (15q26.1), JAK2 (9p24.1), JAK3 (19p13.11), KIT (4q12), KRAS (12p12.1), MAP2K1 (15q22.31), MAP2K2 (19p13.3), MAPK1 (22q11.22), MAPK3 (16p11.2), MET (7q31.2), MLH1 (3p22.2), MPL (1p34.2), MTOR (1p36.22), MYC (8q24.21), NF1 (17q11.2), NFE2L2 (2q31.2), NOTCH1 (9q34.3), NPM1 (5q35.1), NRAS (1p13.2), NTRK1 (1q23.1), NTRK3 (15q25.3), PDGFRA (4q12), PIK3CA (3q26.32), PTEN (10q23.31), PTPN11 (12q24.13), RAF1 (3p25.2), RB1 (13q14.2), RET (10q11.21), RHEB (7q36.1), RHOA (3p21.31), RIT1 (1q22), ROS1 (6q22.1), SMAD4 (18q21.2), SMO (7q32.1), STK11 (19p13.3), TERT (5p15.33), TP53 (17p13.1), TSC1 (9q34.13), and/or VHL (3p25.3). In some embodiments, the protein-coding mutations are in exonic regions of one or more of the following genes: ABL1 ACVR1B AKT1 AKT2 AKT3 ALK ALOX12B AMER1 (FAM123B) APC AR ARAF ARFRP1 ARID1A ASXL1 ATM ATR ATRX AURKA AURKB AXIN1 AXL BAP1 BARD1 BCL2 BCL2L1 BCL2L2 BCL6 BCOR BCORL1 BRAF BRCA1 BRCA2 BRD4 BRIP1 BTG1 BTG2 BTK C11orf30 (EMSY) CALR CARD11 CASP8 CBFB CBL CCND1 CCND2 CCND3 CCNE1 CD22 CD274 (PD-L1) CD70 CD79A CD79B CDC73 CDH1 CDK12 CDK4 CDK6 CDK8 CDKN1A CDKN1B CDKN2A CDKN2B CDKN2C CEBPA CHEK1 CHEK2 CIC CREBBP CRKL CSF1R CSF3R CTCF CTNNA1 CTNNB1 CUL3 CUL4A CXCR4 CYP17A1 DAXX DDR1 DDR2 DIS3 DNMT3A DOT1L EED EGFR EP300 EPHA3 EPHB1 EPHB4 ERBB2 ERBB3 ERBB4 ERCC4 ERG ERRFI1 ESR1 EZH2 FAM46C FANCA FANCC FANCG FANCL FAS FBXW7 FGF10 FGF12 FGF14 FGF19 FGF23 FGF3 FGF4 FGF6 FGFR1 FGFR2 FGFR3 FGFR4 FH FLCN FLT1 FLT3 FOXL2 FUBP1 GABRA6 GATA3 GATA4 GATA6 GID4 (C17orf39) GNA11 GNA13 GNAQ GNAS GRM3 GSK3B H3F3A HDAC1 HGF HNF1A HRAS HSD3B1 ID3 IDH1 IDH2 IGF1R IKBKE IKZF1 INPP4B IRF2 IRF4 IRS2 JAK1 JAK2 JAK3 JUN KDM5A KDM5C KDM6A KDR KEAP1 KEL KIT KLHL6 KMT2A (MLL) KMT2D (MLL2) KRAS LTK LYN MAF MAP2K1 (MEK1) MAP2K2 (MEK2) MAP2K4 MAP3K1 MAP3K13 MAPK1 MCL1 MDM2 MDM4 MED12 MEF2B MEN1 MERTK MET MITF MKNK1 MLH1 MPL MRE11A MSH2 MSH3 MSH6 MST1R MTAP MTOR MUTYH MYC MYCL (MYCL1) MYCN MYD88 NBN NF1 NF2 NFE2L2 NFKBIA NKX2-1 NOTCH1 NOTCH2 NOTCH3 NPM1 NRAS NT5C2 NTRK1 NTRK2 NTRK3 P2RY8 PALB2 PARK2 PARP1 PARP2 PARP3 PAX5 PBRM1 PDCD1 (PD-1) PDCD1LG2 (PD-L2) PDGFRA PDGFRB PDK1 PIK3C2B PIK3C2G PIK3CA PIK3CB PIK3R1 PIM1 PMS2 POLD1 POLE PPARG PPP2R1A PPP2R2A PRDM1 PRKAR1A PRKCI PTCH1 PTEN PTPN11 PTPRO QKI RAC1 RAD21 RAD51 RAD51B RAD51C RAD51D RAD52 RAD54L RAF1 RARA RB1 RBM10 REL RET RICTOR RNF43 ROS1 RPTOR SDHA SDHB SDHC SDHD SETD2 SF3B1 SGK1 SMAD2 SMAD4 SMARCA4 SMARCB1 SMO SNCAIP SOCS1 SOX2 SOX9 SPEN SPOP SRC STAG2 STAT3 STK11 SUFU SYK TBX3 TEK TET2 TGFBR2 TIPARP TNFAIP3 TNFRSF14 TP53 TSC1 TSC2 TYRO3 U2AF1 VEGFA VHL WHSC1 (MMSET) WHSC1L1 WT1 XPO1 XRCC2 ZNF217 ZNF703.

In some embodiments, the protein coding somatic mutations identified using WES from each patient are selected and the most severe consequence of each mutation is retained. Sliding windows with lengths of 8, 9, 10, 11, 12, 13, 14, 15, 16, 17, 18, 19, 20, 21, 22, 23, 24, 25, 26, 27, 28, 29, 30 amino acids are arranged over each mutation and all windows containing the mutation are retained. Each of these mutation-containing amino acid sequences is then paired with each of the patient's MHC-I and MHC-II alleles in all combinations and analyzed with one or more neoantigen and/or neoantigen classifiers.

In some embodiments, the neoantigens are identified using a neoantigen classifier that not only predicts whether a mutation will be displayed on the cell surface as a neo-epitope, but also predicts immunogenicity (i.e., whether a T cell is likely to respond to the neo-epitope). The neoantigen classifiers can predict immunogenicity by examining the physio-chemical properties of the mutant peptide bound to MHC. They are machine learning models trained on experimental datasets composed of neo-epitopes that stimulate T cell activation and other neo-epitopes that did not stimulate immune activation. The additional prediction of immunogenicity by the neoantigen classifier improves immunotherapy response prediction.

In some embodiments, the neoantigens are identified using a plurality of different neoantigen and/or neoantigen classifiers, such as at least 2, at least 3, at least 4, or at least 5 different neoantigen and/or neoantigen classifiers. In some embodiments, the neoantigens are identified using a plurality of different neoantigen classifiers, such as at least 2, at least 3, at least 4, or at least 5 different neoantigen classifiers.

In some embodiments, the neoantigen and/or neoantigen classifiers are capable of evaluating whether the patient's MHC alleles can bind to the mutant amino acid sequences and/or whether the mutant amino acid sequence could stimulate a T-cell response (immunogenicity). The T-cell response may be a CD8+ cell response and/or a CD4+ cell response. Those mutant amino acid sequences that strongly bind to the patient's MHC I alleles and/or are immunogenic are retained (e.g., IC₅₀<600 nM, or IC₅₀<550 nM, or IC₅₀<500 nM, or IC₅₀<450 nM, or IC₅₀<400 nM; and/or percentile rank<0.6%, or percentile rank<0.55%, or percentile rank<0.5%, or percentile rank<0.45%, or percentile rank<0.4%). The number of neoantigens and/or neoantigens is summed for each of the neoantigen and/or neoantigen classifiers.

In some embodiments, RNAseq data are used to assemble T and B-cell receptor sequences present within the tumor biopsy. In some embodiments, the number of unique T- and B-cell receptors (alpha diversity) within the biopsy's RNAseq data is quantified. In some embodiments, tumor gene expression data are used to determine the enrichment of immune cell populations present within the tumor. In some embodiments, mutant amino acid sequences derived from an un-expressed gene are removed.

In some embodiments, quantifying the number of unique T- and B-cell receptors comprises: (i) deconvoluting proportions of immune cells in the tumor sample based on RNA sequencing data, and (ii) assembling B and T-cell receptors to quantify the number of unique T- and B-cell receptors.

In some embodiments, tumor gene expression data are used to determine the enrichment of the following immune cell populations present within the tumor: classical monocytes (Monocytes.C), plasmacytoid dendritic cells (pDCs), or both. In some embodiments, tumor gene expression data are used to determine the enrichment of the following immune cell populations present within the tumor: mucosal associated invariant T-cells (MAIT), myeloid dendritic cells (mDCs), low-density neutrophils (Neutrophils.LD), CD4⁺ memory T-cells (T.CD4 Memory), CD4⁺ Naïve T-cells (T.CD4 Naïve), IFNG single gene expression, CD274 single gene expression, or a combination thereof. In some embodiments, tumor gene expression data are used to determine the enrichment of the following immune cell populations present within the tumor: memory B-cells (B.Memory), naive B-cells (B.Naive), low-density basophils (Basophils.LD), non-classical intermediate monocytes (Monocytes.NC.I), natural killer cells (NK), Plasmablasts, CD8⁺ memory T-cells (T.CD8.Memory), CD8⁺ Naïve T-cells (T.CD8.Naive), non-VD2 gamma delta T-cells (T.gd.non.Vd2), VD2 gamma delta T-cells (T.gd.Vd2), CD8A single gene expression, CD8B single gene expression, CD4 single gene expression, CD8/CD4 expression ratio, TCGA subtype, or a combination thereof.

In some embodiments, RNAseq data are used to determine expression of one or more immune checkpoint molecules in the tumor sample. In some embodiments, the immune checkpoint molecular comprises CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GAL9, TIM-3, TIGHT, VISTA, 2B4, CGEN-15049, CHK 1, CHK2, A2aR, TGF-β, PI3Kγ, GITR, ICOS, IDO, TLR, IL-2R, IL-10, PVRIG, CCRY, OX-40, CD160, CD20, CD52, CD47, CD73, CD27-CD70, CD40, or a combination thereof.

In some embodiments, the RNA sequencing is bulk RNA sequencing. In some embodiments, the RNA sequencing is single-cell RNA sequencing or targeted T and B cell receptor sequencing.

In some embodiments, the tumor sample of the patient is from a solid tumor. In some embodiments, the tumor sample of the patient is from a solid tumor. In some embodiment, the tumor sample of the patient is from a tumor of abdomen or abdominal wall, adrenal gland, anus, appendix, bladder, bone, brain, breast, cervix, chest wall, colon, diaphragm, duodenum, ear, endometrium, esophagus, fallopian tube, gallbladder, gastro-esophageal junction, head and neck, kidney, larynx, liver, lung, lymph node, malignant effusions, mediastinum, nasal cavity, omentum, ovarian, pancreas, pancreatobiliary, parotid gland, pelvis, penis, pericardium, peritoneum, pleura, prostate, rectum, salivary gland, skin, small intestine, soft tissue, spleen, stomach, thyroid, tongue, trachea, ureter, uterus, vagina, vulva, or whipple resection. In some embodiments, the patient suffers from breast cancer, colorectal cancer, gastrointestinal cancer, kidney cancer, lung cancer, bladder cancer, ovarian cancer, or pancreatic cancer.

In some embodiments, a cancer patient is identified as an immunotherapy responder using an analytic model based on the number of neoantigens in the tumor sample, the number and abundance of unique T- and B-cell receptors, and the enrichment of immune cell populations in the tumor sample. In some embodiments, the analytic model further considers pre-immunotherapy ctDNA results and clinical characteristics (e.g., age, cancer stage, previous therapies, metastases). Detection of ctDNA in a blood sample of the cancer patient is described in, for example, WO2015/164432 and WO2019/200228, which are incorporated by reference in their entireties.

In some embodiments, a machine learning model, such as a random forest model, is trained using the number of neoantigens in the tumor sample, the number of unique T- and B-cell receptors, and the enrichment of immune cell populations in the tumor sample, pre-immunotherapy ctDNA results and clinical characteristics (e.g., age, cancer stage, previous therapies, metastases). Training of the random forest model was conducted using 80% of patient datasets. Testing of this trained model was conducted using the remaining 20% of the patient datasets. In other examples, the machine learning model may be linear regression, logistic regression, random forest classifiers, cutoffs learned from the historical data, support vector machines, neural networks, or deep neural networks.

In some embodiments, a cancer patient is identified as an immunotherapy responder using the number of neoantigens in the tumor sample the number of unique T- and B-cell receptors, the enrichment of classical monocyte (monocytes C) and plasmacytoid dendritic cell (pDCs) populations in the tumor sample, and optionally the pre-immunotherapy ctDNA-positivity status in a blood sample of the patient.

In some embodiments, a cancer patient is identified as an immunotherapy responder using the number of neoantigens in the tumor sample according to, the number of unique T- and B-cell receptors, the enrichment of classical monocyte (monocytes C), plasmacytoid dendritic cell (pDCs), mucosal associated invariant T-cell (MAIT), myeloid dendritic cell (mDCs), low-density neutrophil (Neutrophils.LD), CD4⁺ memory T-cell (T.CD4 Memory), CD4⁺ naïve T-cell (T.CD4 Naïve) populations, IFNG single gene expression, and CD274 single gene expression in the tumor sample, and optionally the pre-immunotherapy ctDNA-positivity status in a blood sample of the patient.

In some embodiments, a cancer patient is identified as an immunotherapy responder using the number of neoantigens in the tumor sample according to the number of unique T- and B-cell receptors, the enrichment of classical monocytes (monocytes C), plasmacytoid dendritic cells (pDCs), mucosal associated invariant T-cells (MAIT), myeloid dendritic cells (mDCs), low-density neutrophils (Neutrophils.LD), CD4⁺ memory T-cells (T.CD4 Memory), CD4⁺ naïve T-cell (T.CD4 Naïve) populations, IFNG single gene expression, and CD274 single gene expression in the tumor sample, nodal status, baseline ECOG, and optionally the pre-immunotherapy ctDNA-positivity status in a blood sample of the patient.

In some embodiments, the method described herein identifies a cancer patient as an immunotherapy responder without TMB analysis. In some embodiments, the method described herein is free of any step of determining TMB.

In some embodiments, the method identifies a cancer patient as an immunotherapy responder with a positive predictive value (PPV) that is at least 10%, at least 20%, at least 30%, at least 40%, or at least 50% higher than use of TMB alone. In some embodiments, the method identifies a cancer patient as an immunotherapy non-responder with a negative predictive value (NPV) that is at least 10%, at least 20%, at least 30%, at least 40%, or at least 50% higher than use of TMB alone.

In some embodiments, the method identifies a cancer patient as an immunotherapy responder with a sensitivity that is at least 10%, at least 20%, at least 30%, at least 40%, or at least 50% higher than use of TMB alone. In some embodiments, the method identifies a cancer patient as an immunotherapy non-responder with a specificity that is at least 10%, at least 20%, at least 30%, at least 40%, or at least 50% higher than use of TMB alone.

In a further aspect, the present disclosure relates to a method identifying a cancer patient as a PD-1 or PD-L1 or CTLA-4 immunotherapy responder, comprising: performing whole exome sequencing on a tumor sample of the patient to quantify the number of neoantigens in the tumor sample; performing RNA sequencing on a tumor sample of the patient to quantify the number and abundance of unique T- and B-cell receptors, enrichment of immune cell populations in the tumor sample, and expression of PD-1 or PD-L1 or CTLA-4; and identifying a cancer patient as an PD-1 or PD-L1 or CTLA-4 immunotherapy responder using the number of neoantigens in the tumor sample, the number of unique T- and B-cell receptors, the enrichment of immune cell populations in the tumor sample, and the expression of PD-1 or PD-L1 or CTLA-4.

In some embodiments, a cancer patient is identified as a PD-1 or PD-L1 or CTLA-4 immunotherapy responder using the number of neoantigens in the tumor sample, the number and abundance of unique T- and B-cell receptors, the enrichment of classical monocyte (monocytes C) and plasmacytoid dendritic cell (pDCs) populations in the tumor sample, and optionally the pre-immunotherapy ctDNA-positivity status and the expression of PD-1 or PD-L1 in the tumor sample.

In some embodiments, a cancer patient is identified as a PD-1 or PD-L1 or CTLA-4 immunotherapy responder using the number of neoantigens in the tumor sample, the number of unique T- and B-cell receptors, the enrichment of classical monocytes (monocytes C), plasmacytoid dendritic cell (pDCs), mucosal associated invariant T-cells (MAIT), myeloid dendritic cell (mDCs), low-density neutrophil (Neutrophils.LD), CD4+ memory T-cell (T.CD4 Memory), CD4+ naïve T-cell (T.CD4 Naïve) populations, IFNG single gene expression, and CD274 single gene expression in the tumor sample, and optionally the pre-immunotherapy ctDNA-positivity status and the expression of PD-1 or PD-L1 in the tumor sample.

In some embodiments, a cancer patient is identified as a PD-1 or PD-L1 or CTLA-4 immunotherapy responder using the number of neoantigens in the tumor sample, the number and abundance of unique T- and B-cell receptors, the enrichment of classical monocyte (monocytes C), plasmacytoid dendritic cell (pDCs), mucosal associated invariant T-cell (MAIT), myeloid dendritic cell (mDCs), low-density neutrophil (Neutrophils.LD), CD4⁺ memory T-cell (T.CD4 Memory), CD4⁺ naïve T-cell (T.CD4 Naïve) populations, IFNG single gene expression, and CD274 single gene expression in the tumor sample, nodal status, baseline ECOG, and optionally the pre-immunotherapy ctDNA-positivity status and the expression of PD-1 or PD-L1 or CTLA-4 in the tumor sample.

In some embodiments, the method described herein identifies a cancer patient as a PD-1 or PD-L1 or CTLA-4 immunotherapy responder without TMB analysis. In some embodiments, the method described herein is free of any step of determining TMB.

In some embodiments, the term “cancer” refers to or describe the physiological condition in an animal or human that is typically characterized by unregulated cell growth.

In some embodiments, a “tumor” comprises one or more cancerous cells. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord. In some embodiments, the cancer has metastasized. In some embodiments, the cancer has not metastasized.

### Methods for Treating Cancer Using Immunotherapy

The present disclosure further relates to a method for treating cancer, comprising identifying a cancer patient as an immunotherapy responder by the method described herein, and administering an effective amount of an immunotherapy to the cancer patient.

In some embodiments, the immunotherapy comprises a checkpoint inhibitor, a CAR-T therapy, a TCR-T therapy, a NK cell therapy, a neoantigen vaccines, an oncolytic virus, a cytokine, a monoclonal antibody, or a combination thereof.

In some embodiments, the immunotherapy comprises an immune checkpoint inhibitor. The immune checkpoint molecular may be CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GAL9, TIM-3, TIGHT, VISTA, 2B4, CGEN-15049, CHK 1, CHK2, A2aR, TGF-B, PI3Ky, GITR, ICOS, IDO, TLR, IL-2R, IL-10, PVRIG, CCRY, OX-40, CD160, CD20, CD52, CD47, CD73, CD27-CD70, CD40, or a combination thereof. In some embodiments, the immunotherapy comprises an immune checkpoint inhibitor selected from a PD-1 inhibitor, a PD-L1 inhibitor, a CLTA-4 inhibitor, or a combination thereof.

In some embodiment, the immunotherapy comprises Pembrolizumab, Nivolumab, Cemiplimab, Dostarlimab, Atezolizumab, Avelumab, Durvalumab, Ipilimumab, or Tremelimumab. In some embodiment, the immunotherapy comprises Vopratelimab, Spartalizumab, Camrelizumab, Sintilimab, Tislelizumab, Toripalimab, INCMGA00012, AMP-224, AMP-514, KN035, Cosibelimab, AUNP12, CA-170, or BMS-986189.

In some embodiments, the immunotherapy comprises a therapeutic cell composition. Exemplary therapeutic cell compositions include, but are not limited to T cells, natural killer (NK) cells and dendritic cells, chimeric antigen receptor T (CAR-T) cells, T-cell receptor-engineered T (TCR-T) cells, and chimeric antigen receptor-natural killer (CAR-NK) cells. In some embodiments, the immunotherapy comprises a therapeutic cell composition in combination with a cancer vaccine.

The present disclosure further relates to a method for treating cancer, comprising identifying a cancer patient as a PD-1 or PD-L1 or CTLA-4 immunotherapy responder by the method described herein, and administering an effective amount of an immunotherapy to the cancer patient, wherein the PD-1 or PD-L1 or CTLA-4 immunotherapy comprises Pembrolizumab, Nivolumab, Cemiplimab, Dostarlimab, Atezolizumab, Avelumab, Durvalumab, Ipilimumab, or Tremelimumab.

In some embodiments, the cancer patient has been or is concurrently treated with surgery, chemotherapy, or radiation therapy.

In some embodiments, the cancer is breast cancer, colorectal cancer, gastrointestinal cancer, kidney cancer, lung cancer, bladder cancer, ovarian cancer, or pancreatic cancer.

In some embodiments, the cancer is a cancer or tumor of abdomen or abdominal wall, adrenal gland, anus, appendix, bladder, bone, brain, breast, cervix, chest wall, colon, diaphragm, duodenum, ear, endometrium, esophagus, fallopian tube, gallbladder, gastro-esophageal junction, head and neck, kidney, larynx, liver, lung, lymph node, malignant effusions, mediastinum, nasal cavity, omentum, ovarian, pancreas, pancreatobiliary, parotid gland, pelvis, penis, pericardium, peritoneum, pleura, prostate, rectum, salivary gland, skin, small intestine, soft tissue, spleen, stomach, thyroid, tongue, trachea, ureter, uterus, vagina, vulva, or whipple resection.

Exemplary cancers for any of the methods described herein include solid tumors, carcinomas, sarcomas, lymphomas, leukemias, germ cell tumors, or blastomas. In some embodiments, the cancer is an acute lymphoblastic leukemia, acute myeloid leukemia, adrenocortical carcinoma, anal cancer, appendix cancer, astrocytoma (such as childhood cerebellar or cerebral astrocytoma), basal-cell carcinoma, bile duct cancer (such as extrahepatic bile duct cancer) bladder cancer, bone tumor (such as osteosarcoma or malignant fibrous histiocytoma), brainstem glioma, brain cancer (such as cerebellar astrocytoma, cerebral astrocytoma/malignant glioma, ependymo, medulloblastoma, supratentorial primitive neuroectodermal tumors, or visual pathway and hypothalamic glioma), glioblastoma, breast cancer, bronchial adenoma or carcinoid, burkitt's lymphoma, carcinoid tumor (such as a childhood or gastrointestinal carcinoid tumor), carcinoma central nervous system lymphoma, cerebellar astrocytoma or malignant glioma (such as childhood cerebellar astrocytoma or malignant glioma), cervical cancer, childhood cancer, chronic lymphocytic leukemia, chronic myelogenous leukemia, chronic myeloproliferative disorders, colon cancer, cutaneous t-cell lymphoma, desmoplastic small round cell tumor, endometrial cancer, ependymoma, esophageal cancer, ewing's sarcoma, tumor in the ewing family of tumors, extracranial germ cell tumor (such as a childhood extracranial germ cell tumor), extragonadal germ cell tumor, eye cancer (such as intraocular melanoma or retinoblastoma eye cancer), gallbladder cancer, gastric cancer, gastrointestinal carcinoid tumor, gastrointestinal stromal tumor, germ cell tumor (such as extracranial, extragonadal, or ovarian germ cell tumor), gestational trophoblastic tumor, glioma (such as brain stem, childhood cerebral astrocytoma, or childhood visual pathway and hypothalamic glioma), gastric carcinoid, hairy cell leukemia, head and neck cancer, heart cancer, hepatocellular (liver) cancer, hodgkin lymphoma, hypopharyngeal cancer, hypothalamic and visual pathway glioma (such as childhood visual pathway glioma), islet cell carcinoma (such as endocrine or pancreas islet cell carcinoma), kaposi sarcoma, kidney cancer, laryngeal cancer, leukemia (such as acute lymphoblastic, acute myeloid, chronic lymphocytic, chronic myelogenous, or hairy cell leukemia), lip or oral cavity cancer, liposarcoma, liver cancer (such as non-small cell or small cell cancer), lung cancer, lymphoma (such as AIDS-related, burkitt, cutaneous T cell, Hodgkin, non-hodgkin, or central nervous system lymphoma), macroglobulinemia (such as waldenstrom macroglobulinemia, malignant fibrous histiocytoma of bone or osteosarcoma, medulloblastoma (such as childhood medulloblastoma), melanoma, merkel cell carcinoma, mesothelioma (such as adult or childhood mesothelioma), metastatic squamous neck cancer with occult, mouth cancer, multiple endocrine neoplasia syndrome (such as childhood multiple endocrine neoplasia syndrome), multiple myeloma or plasma cell neoplasm, mycosis fungoides, myelodysplastic syndrome, myelodysplastic or myeloproliferative disease, myelogenous leukemia (such as chronic myelogenous leukemia), myeloid leukemia (such as adult acute or childhood acute myeloid leukemia), myeloproliferative disorder (such as chronic myeloproliferative disorder), nasal cavity or paranasal sinus cancer, nasopharyngeal carcinoma, neuroblastoma, oral cancer, oropharyngeal cancer, osteosarcoma or malignant fibrous histiocytoma of bone, ovarian cancer, ovarian epithelial cancer, ovarian germ cell tumor, ovarian low malignant potential tumor, pancreatic cancer (such as islet cell pancreatic cancer), paranasal sinus or nasal cavity cancer, parathyroid cancer, penile cancer, pharyngeal cancer, pheochromocytoma, pineal astrocytoma, pineal germinoma, pineoblastoma or supratentorial primitive neuroectodermal tumor (such as childhood pineoblastoma or supratentorial primitive neuroectodermal tumor), pituitary adenoma, plasma cell neoplasia, pleuropulmonary blastoma, primary central nervous system lymphoma, cancer, rectal cancer, renal cell carcinoma, renal pelvis or ureter cancer (such as renal pelvis or ureter transitional cell cancer, retinoblastoma, rhabdomyosarcoma (such as childhood rhabdomyosarcoma), salivary gland cancer, sarcoma (such as sarcoma in the ewing family of tumors, Kaposi, soft tissue, or uterine sarcoma), sézary syndrome, skin cancer (such as nonmelanoma, melanoma, or merkel cell skin cancer), small intestine cancer, squamous cell carcinoma, supratentorial primitive neuroectodermal tumor (such as childhood supratentorial primitive neuroectodermal tumor), T-cell lymphoma (such as cutaneous T-cell lymphoma), testicular cancer, throat cancer, thymoma (such as childhood thymoma), thymoma or thymic carcinoma, thyroid cancer (such as childhood thyroid cancer), trophoblastic tumor (such as gestational trophoblastic tumor), unknown primary site carcinoma (such as adult or childhood unknown primary site carcinoma), urethral cancer (such as endometrial uterine cancer), uterine sarcoma, vaginal cancer, visual pathway or hypothalamic glioma (such as childhood visual pathway or hypothalamic glioma), vulvar cancer, waldenström macroglobulinemia, or wilms tumor (such as childhood wilms tumor).

A machine learning tool is provided to predict individual response to immune checkpoint inhibitor (ICI) treatment based on a large cohort of individuals with complete clinical information. By utilizing whole exome sequencing data, ctDNA time point analyses, and clinical data, a deep learning immune response prediction model successfully predicts which individuals who respond to and benefit from ICI treatment across multiple cancer types.

Examples herein utilize ctDNA status and/or neoantigen burden for risk stratification and treatment response prediction and their use in conjunction with biomarker/clinicopathologic features to significantly improve treatment strategies. The development of such multifactorial models require large individual cohorts with detailed clinical information. The immune response prediction model was developed using comprehensive and wide-ranging data sets that a machine learning algorithm evaluated and identified the optimal set of features that can accurately predict individual response to ICI therapy. This immune response prediction model was tested and validated in individuals with melanoma, NSCLC, CRC, and validation in additional cancer types for which ICI therapy is a potential line of treatment. Because this immune response prediction model was based on accessible genomic and clinicopathological data that is collected as a part of routine development of tumor-informed, bespoke ctDNA assays and of evaluation of tumor drive variants via comprehensive genome profiling, the immune response prediction model may be utilized at the beginning of treatment and ctDNA assay development in individuals with tumors that may benefit from ICI therapy.

### Example 1

Predictive biomarkers can help enrich individuals who are most likely to benefit from immune checkpoint inhibitors (ICIs), but predictive accuracy and specificity are limited. An immune response prediction model is provided that includes neoantigen load and clinicopathologic variables to improve the prediction of ICI response when compared with tumor mutational burden (TMB).

The immune response prediction model is based on tumor and matched normal whole exome sequencing data from individuals with solid tumors (melanoma n=238, non-small cell lung cancer (NSCLC) n=133, high microsatellite instable colorectal cancer (MSI-high CRC) n=172) who received ICI and were referred for a personalized, tumor-informed mPCR-NGS circulating tumor (ct) DNA assay. The model was then trained using predicted neoantigens based on several open-source classifiers and clinicopathological characteristics in 80% of individuals. Progression-free survival (PFS) following ICI was used to evaluate the performance of the model versus TMB status alone in the remaining 20% of individuals and an independent validation set of individuals enrolled in the BESPOKE IO clinical trial (NCT04761783).

The immune response prediction model selected parameters including neoantigen predictors, nodal status, baseline ECOG score, and pre-ICI ctDNA. In melanoma individuals, both the model and TMB predicted response, though the model (HR=12.68, P<0.001, area under the curve (AUC)=0.94) was more accurate compared to TMB (HR=3.23, P=0.021, AUC=0.82). Among NSCLC individuals, the model predicted PFS better than TMB (Model: HR=6.73 P=0.006, AUC=0.88; TMB: HR=1.31, P=0.69, AUC=0.64) and a similar trend was observed among MSI-high CRC individuals (Model: HR=6.10, P=0.014, AUC=0.85; TMB: HR=3.52, P=0.076, AUC=0.5). These results were further confirmed in the validation BESPOKE IO dataset (Model: HR=3.062, P<0.001, AUC=0.75; TMB: HR=1.76, P=0.094, AUC=0.70). Incorporation of neoantigen load and other clinical variables significantly improved the prediction of ICI response in individuals compared to TMB. The model may be utilized with a variety of different cancer types.

### Study Design and Clinical Data Abstraction

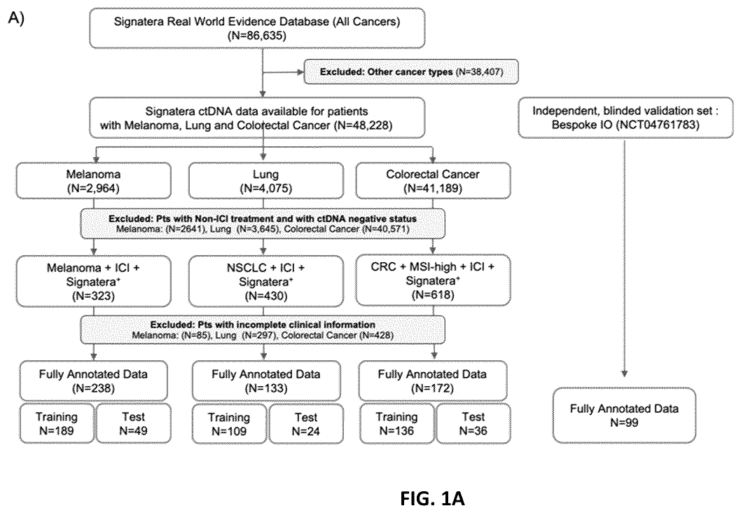

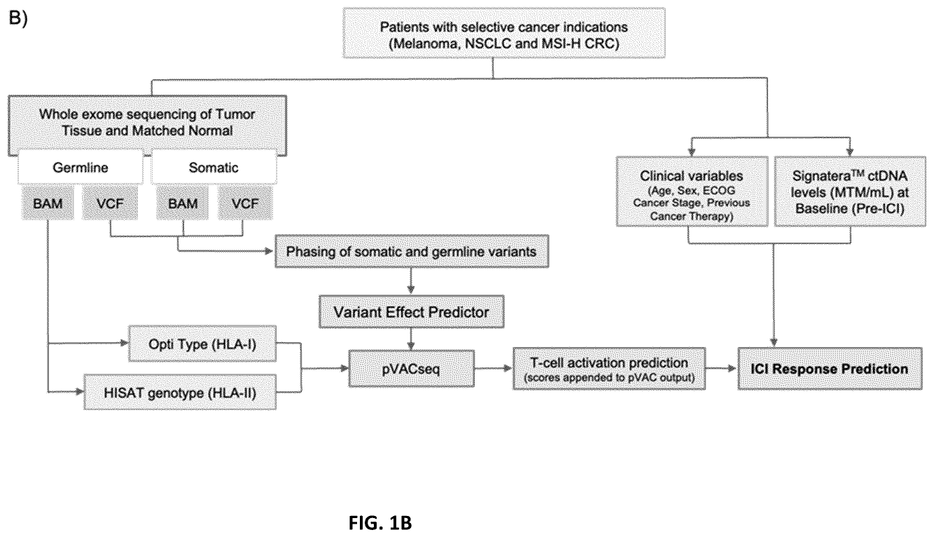

To develop an immune response prediction model, a retrospective analysis was performed on whole exome sequencing (WES) data sourced from a clinical genomics database (N=86,635) as shown in FIG. 1A. FIG. 1A is diagram of individual inclusion in sub analysis. WES was performed as part of ctDNA testing. Analysis included individuals with malignant melanoma, non-small cell lung cancer (NSCLC), and colorectal cancer (CRC), which accounted for a total of 48,228 individuals. Clinical outcomes of individuals who have undergone clinically indicated testing are collected as a part of ongoing quality assurance operations. Individuals who received ICI treatments without other concurrent therapies and had a ctDNA positive test any time at baseline (pre-ICI) or after initiation of ICI, and for whom complete clinical outcomes information was available in the clinical testing database were included in the real-world data analysis (melanoma: 238, NSCLC: 133, MSI-high CRC: 172) as shown in FIG. 1A. De-identified individual information including age, sex, ECOG score, cancer type, and stage, was abstracted and included in these analyses as per IRB-approved Quality Assurance protocol (Salus Protocol #20099-01). The study was conducted in accordance with the Declaration of Helsinki.

In addition, a blinded validation cohort of 99 individuals with lung, melanoma, and MSI-high CRC who were enrolled in a prospective, longitudinal, multicenter observational study (BESPOKE IO, https://clinicaltrials.gov/study/NCT04761783, approved by the ethical and independent review services protocol #Natera-20-043-NCP BESPOKE Study of ctDNA Guided Immunotherapy) were included in the analysis. The cohort included here represents an interim set of individuals with fully annotated data and a median of 10 months of clinical follow-up.

### WES DNA Analysis

FIG. 1B is a schematic of an exemplary immune response prediction model. FIG. 1B is a representation of the process involved in developing the machine learning-based response prediction model. For each individual, WES data was processed from FFPE tumor tissue and matched normal blood for somatic and germline genomic information (green boxes), which was used to phase variants and was used as an input to the variant effect predictor and to genotype MHC-I and MHC-II alleles. These data were evaluated for the MHC-peptide binding prediction and T-cell activation prediction. This data, in addition to clinical variables and ctDNA results, were included in the immune response prediction model.

Formalin-fixed and paraffin-embedded tumor tissues and matched normal blood samples were subjected to WES and bioinformatically processed. Tumor and germline consensus single nucleotide variants (SNVs) and insertions/deletions (indels) were identified via calling algorithms using the aligned tumor and normal BAM files. Somatic SNVs and indels were phased with germline variants to determine mutation-germline allele haplotypes and then annotated (VEP v109) to determine the highest impact consequence of each mutation. MHC class-I (MHC-I) alleles were genotyped using OptiType v1.3.3. MHC class-II (MHC-II) alleles were genotyped using HISATgenotype v1.3.3.

### Peptide-MHC Binding Prediction

Peptide-MHC binding prediction was conducted in two steps. First, peptide-MHC binding metrics were summarized using all detected amino-acid sequence altering mutations and MHC-I and MHC-II alleles using pVAC-Seq v4.0.5 and subsequent MHC-I-mutant peptide processing and presentation scores were predicted using MHCflurry v2.1 and MHCflurryEL v2.1. Next, the prediction of T-cell immunogenicity for each mutant peptide-MHC-I combination was evaluated as shown in FIG. 1B.

### Personalized, Tumor-Informed ctDNA Assay

Briefly, in order to develop personalized assays, 16 individual-specific, somatic single nucleotide variants (SNVs) were selected from the WES tumor and matched normal data to design individual-specific primers. Plasma samples isolated from whole blood were then analyzed for ctDNA detection. Plasma samples with at least 2 out of 16 variants detected above a predefined algorithmic confidence threshold were defined as ctDNA-positive and the concentration was measured and reported in mean tumor molecules (MTM)/mL of plasma.

### Tumor Mutational Burden

TMB was calculated as the number of non-synonymous somatic mutations identified via WES divided by the WES hybrid-capture panel size in mega-bases. TMB values≥10 mutations/Mb were considered ‘high,’ while TMB values<10 mutations/Mb were considered ‘low’.

### Univariate Analyses of Clinicopathologic Characteristics

The effect of individual age (above/below 65 years), sex, ECOG score, cancer stage, pre-ICI ctDNA results (positive/negative), and TMB (above/below 10 non-synonymous mutations/Mb) on progression free survival (PFS) were individually tested with Cox proportional hazards with the R v4.2.2 library survival v3.4. PFS was measured from the date of ICI-initiation to the first documented sign of radiological progression, with data censored at the last follow-up or death. Forest plots were generated using the R library survival Analysis v0.3.0.

### Model Training and Testing

Immunogenicity of neoantigens varies across a spectrum of characteristics/features and cannot be captured with a single score. Here, each individual's set of neoantigens is cumulatively scored across all combinations of five properties: processing (0-0.025, 0.025-0.05, 0.05-0.1, 0.1-0.5, 0.5 0.5-1), presentation (0-0.1, 0.1-0.2, 0.2-0.4, 0.4-0.6, 0.6-1), MHC binding fold change as measured by log2 (WT IC50/MT IC50) (0-1, 1-2, 2-3, 3-4, 4-5, >5), predicted immunogenicity based on percent rank (0-0.25, 0.25-0.5, 0.5-1, 1-5, 5-10, >10), and dissimilarity from the reference human proteome based on (<0.75, ≥0.75).

A random survival forest model was then trained to predict cancer progression using the above data, pre-ICI ctDNA results, and clinical characteristics (e.g., age, sex, ECOG score, cancer stage). Training was conducted using 80% of the individuals while testing was conducted using the remaining 20% of individuals. Training was conducted using 5-fold cross-validation and the optimal model was chosen based on the integrated Brier score. The training and test datasets were balanced to include equal proportions of individuals who had progressed. Analyses were conducted using R v4.2.2, and the tidymodels v1.0.0 and partykit 1.2-20 libraries. KM curves were plotted using the R survminer v0.4.9 library; AUC was calculated using the pROC v1.18.5 library.

### Results

Altogether, 543 individuals with selective cancer indications were included in the training and test set as shown in FIG. 1A. These individuals included 238 (43.8%) individuals diagnosed with melanoma, 133 (24.5%) with NSCLC, and 172 (31.7%) with MSI-high CRC. An additional 99 individuals with solid tumors were included in a validation set. The most common ICI regimen varied based on cancer type: ipilimumab plus nivolumab for melanoma individuals (33.9%), pembrolizumab for NSCLC (47.9%) and CRC (70.0%) individuals, and ipilimumab plus nivolumab for Bespoke IO individuals (37.4%). Median PFS was 161 days for melanoma, 253 days for NSCLC, 259 days for MSI-high CRC, and 148 days for the BESPOKE IO validation dataset. Full demographic information is included below in Table 1.

TABLE 1
Individual demographic and clinical characteristics

| | | | | Blinded |
|---|---|---|---|---|
| | Melanoma | Lung | MSI-high CRC | Validation Cohort |
| | (N = 238) | (N = 133) | (N = 172) | (N = 99) |
| Median Age | 63.5 (20-91) | 68.6 (39-87) | 66.0 (21-92) | 67.0 (23-88) |
| (Range) | | | | |
| Sex | | | | |
| Female | 37.0% | 49.0% | 58.1% | 48.5% |
| Male | 63.0% | 51.0% | 41.9% | 51.5% |
| Median ECOG | 0 | 1 | 1 | 1 |
| score | | | | |
| Clinical Stage | | | | |
| I | 6.0% | 2.6% | 1.6% | 3.4% |
| II | 8.7% | 5.2% | 9.5% | 9.0% |
| III | 33.2% | 28.1% | 35.8% | 37.1% |
| IV | 52.2% | 64.0% | 53.2% | 50.6% |
| Type of ICI, % | Ipilimumab And | Pembrolizumab (47.9%) | Pembrolizumab (70.0%) | Ipilimumab and |
| | Nivolumab (33.9%) | Durvalumab (19.0%) | Nivolumab (13.7%) | Nivolumab (37.4%) |
| | Pembrolizumab (31.9%) | Ipilimumab And | Ipilimumab and | Pembrolizumab (27.3%) |
| | Nivolumab (16.3%) | Nivolumab (16.2%) | Nivolumab (13.7%) | Durvalumab (21.2%) |
| | nivolumab and | Nivolumab (11.3%) | Dostarlimab (2.1%) | Nivolumab (8.1%) |
| | opdualag (15.6%) | Atezolizumab (5.6%) | Atezolizumab (0.5%) | Nivolumab and |
| | Ipilimumab (1.6%) | | | Pembrolizumab (2.0%) |
| | Tebentafusp (0.8%) | | | Nivolumab and |
| | Nivo + | | | Pembrolizumab and |
| | Relatlimab (0.8%) | | | Ipilimumab (2.0%) |
| | | | | Atezolizumab (1.0%) |
| | | | | Cemiplimab (1.0%) |
| Baseline ctDNA | | | | |
| detection rates | | | | |
| I | 72.3% | 100.0% | 100.0% | 33.3% |
| II | 87.5% | 71.4% | 77.0% | 42.9% |
| III | 65.6% | 61.9% | 93.2% | 56.7% |
| IV | 85.9% | 70.2% | 90.2% | 91.4% |
| Response to ICI | | | | |
| Responders % | 68.5% | 64.7% | 77.0% | 58.6% |
| Non-responders % | 31.5% | 35.3% | 23.0% | 41.4% |

### Melanoma

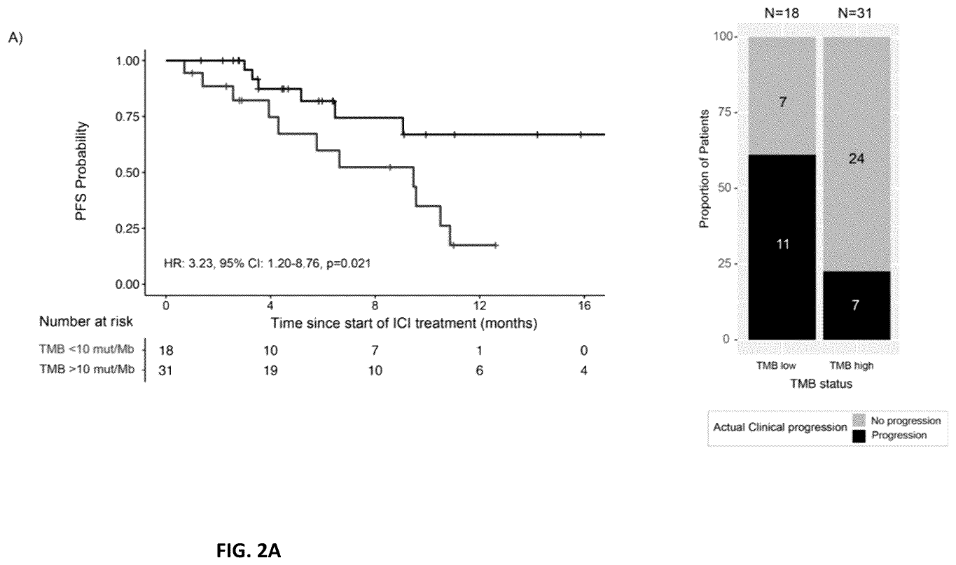

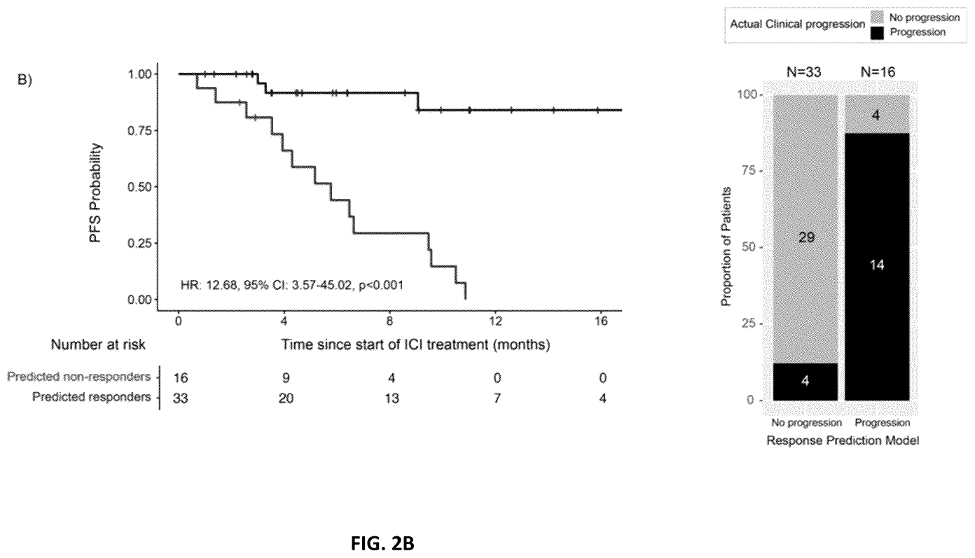

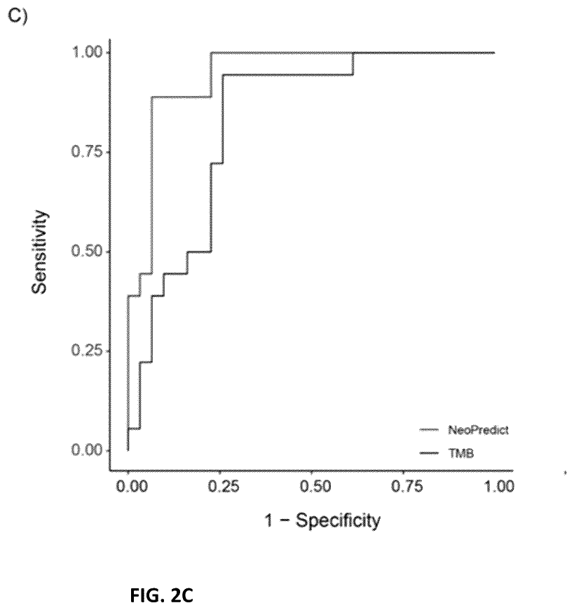

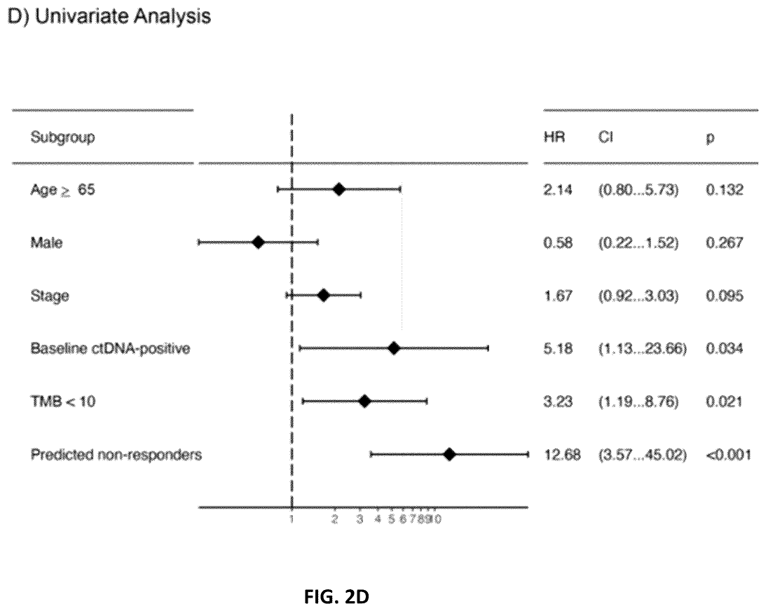

FIG. 2A is a graphical representation of Kaplan-Meir estimates of progression-free survival for 49 individuals with melanoma stratified by TMB status. The median follow-up period for melanoma individuals (N=49) was 6 months (range 1-62 months). When evaluating the predictive value of TMB, progression was reported for 22.6% (7 out of 31) of individuals with TMB>10 mut/Mb compared with 61.1% (11 out of 18) of individuals with TMB<10 mut/Mb (HR: 3.23, 95% Cl 1.20-8.76, P=0.021) as shown in FIG. 2A.

FIG. 2B is a graphical representation of Kaplan-Meir estimates of progression-free survival for 49 individuals with melanoma stratified based on response prediction model. When evaluating the predictive value of the model, progression was reported for 87.5% (14 out of 16) of non-responders (individuals with predicted progression) compared with 12.1% (4 out of 33) of responders (individuals without predicted progression) (HR: 12.68, 95% Cl 3.57-45.02, P<0.001) as shown in FIG. 2B.

FIG. 2C is a graphical representation of the area under the curve (AUC) for prediction of ICI response for TMB and prediction model. The predictive accuracies of both TMB and the model were computed and yielded an AUC of 0.82 for TMB alone and 0.94 for the model as shown in FIG. 2C. FIG. 2D is a graphical representation univariate analysis to assess the predictive value of the immune response prediction model and other clinicopathological factors. Among other clinicopathological features, the immune response prediction model was the most significant factor predictive of increased risk for progression (p<0.001) in univariate analysis, followed by baseline positivity (p=0.034) and TMB status (p=0.021) as shown in FIG. 2D. Survival analyses were performed using the Kaplan-Meier Estimator and the Cox method.

### NSCLC

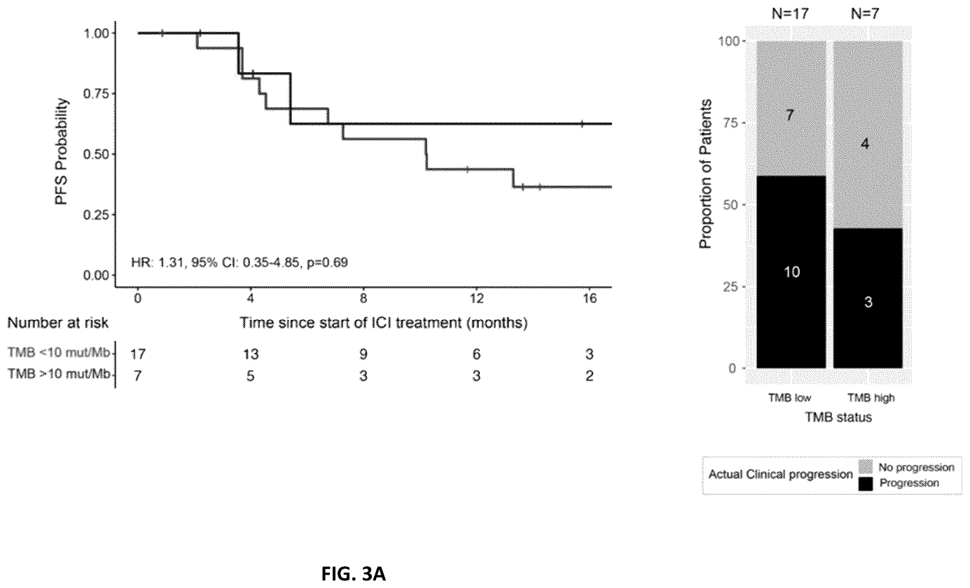

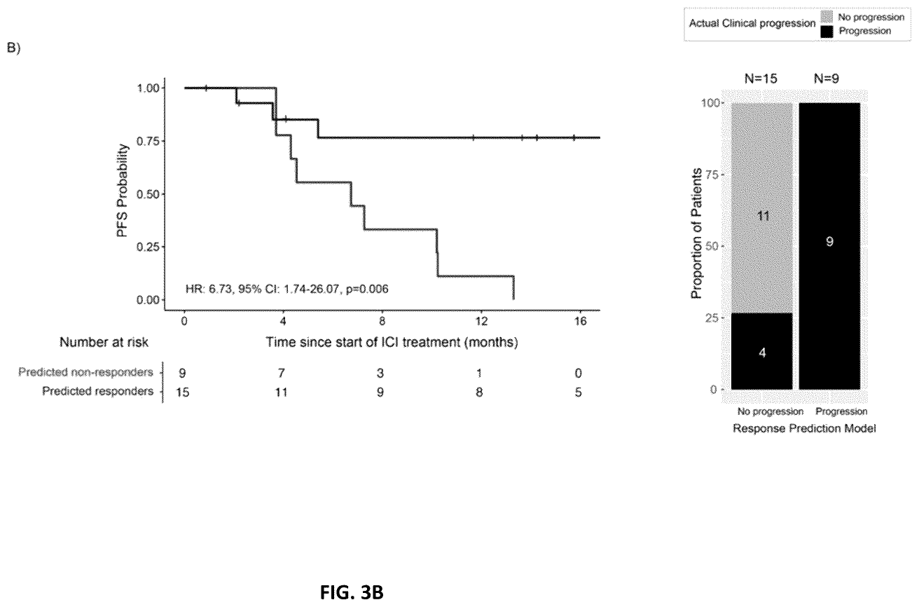

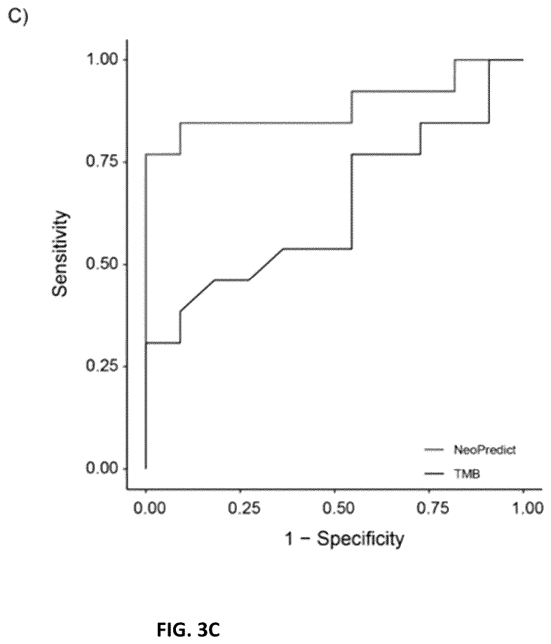

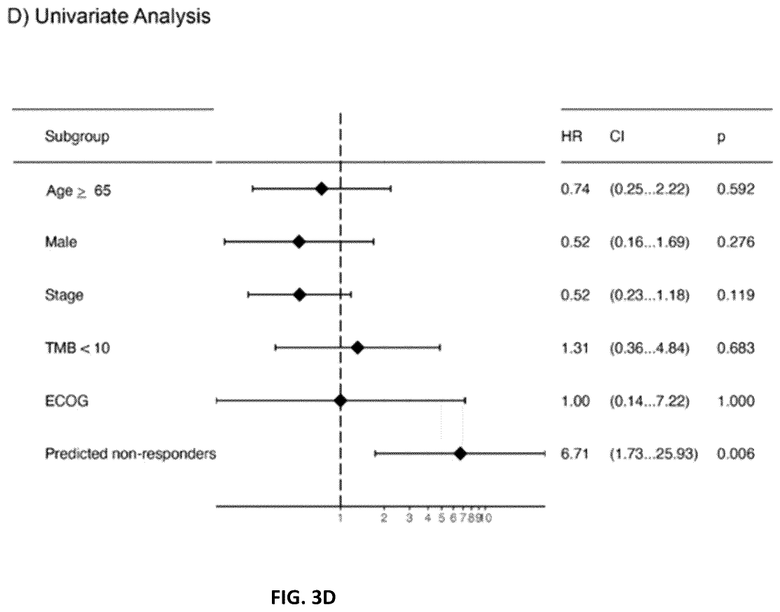

FIG. 3A is a graphical representation of Kaplan-Meir estimates of progression-free survival for 24 individuals with NSCLC stratified by TMB status. The median follow-up period for NSCLC individuals was 12 months (range 1-82 months). When evaluating the predictive value of TMB, progression was reported for 42.9% (3 out of 7) of individuals with TMB>10 mut/Mb compared with 58.8% (10 out of 17) of those with TMB<10 mut/Mb (HR: 1.31, 95% Cl 0.35-4.85, P=0.69) as shown in FIG. 3A.

FIG. 3B is a graphical representation of Kaplan-Meir estimates of progression-free survival for 24 individuals with NSCLC stratified based on response prediction model. When evaluating the predictive value of the model, progression was reported for 100% (9 out of 9) of non-responders compared with 26.7% (4 out of 15) of non-responders (HR: 6.73 95% CI 1.74-26.07, P=0.006) as shown in FIG. 3B.

FIG. 3C is a graphical representation of the area under the curve (AUC) for prediction of ICI response for TMB and prediction model. The predictive accuracies of both TMB and the model were computed and yielded an AUC of 0.64 for TMB alone and 0.88 for the model as shown in FIG. 3C. FIG. 3D is a graphical representation of univariate analysis to assess the predictive value of the immune response prediction model and other clinicopathological factors. Among other clinicopathological features, the immune response prediction model was the only significant factor predictive of increased risk for progression in univariate analysis (p=0.006) as shown in FIG. 3D. Survival analyses were performed using the Kaplan-Meier Estimator and the Cox method.

### CRC

As ICI treatment is indicated only for CRC individuals with MSI-high tumors, individuals meeting this criteria were included in this analysis. The median follow-up period for melanoma individuals was 14 months (range 2-37 months).

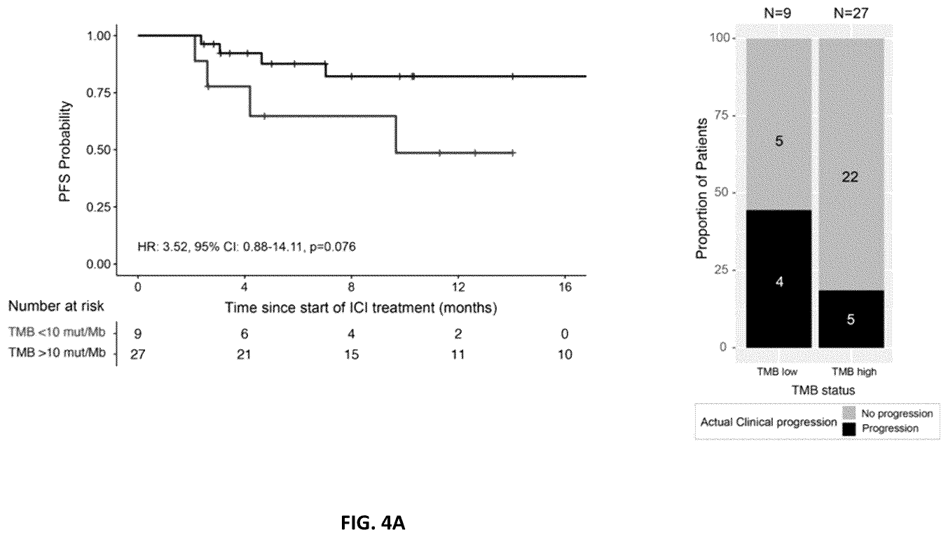

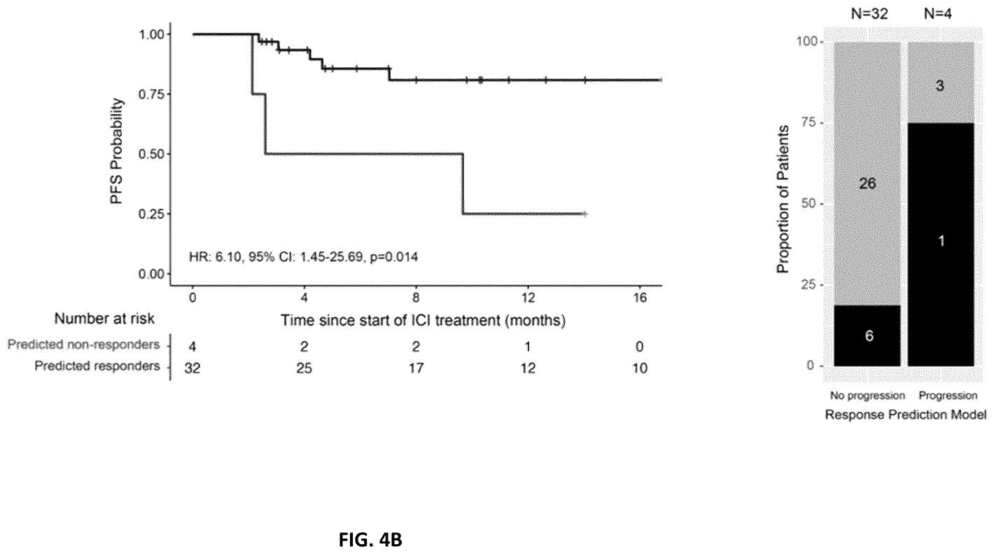

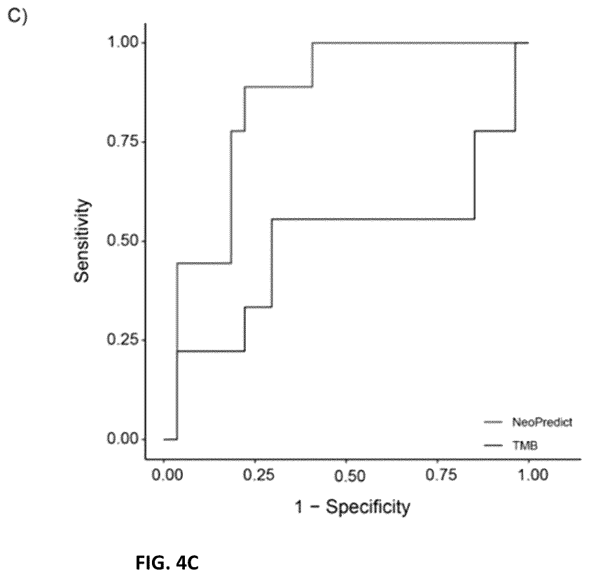

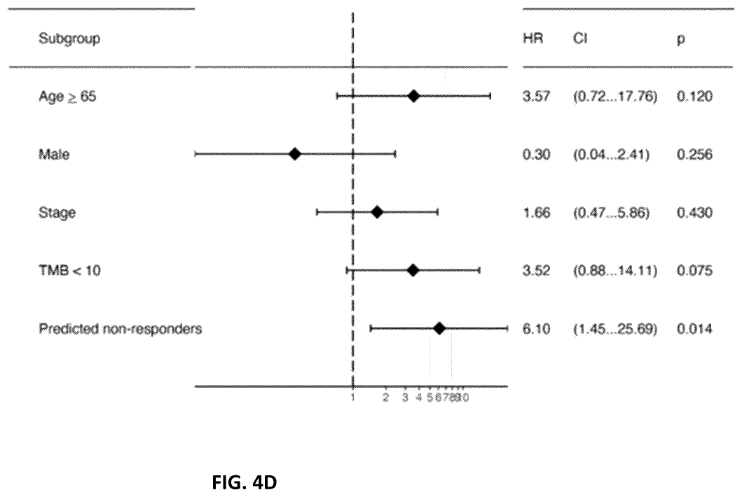

FIG. 4A is a graphical representation of progression-free survival for 36 individuals with MSI-H CRC stratified by TMB status. When evaluating the predictive value of TMB, progression was reported for 18.5% (5 out of 27) of individuals with TMB>10 mut/Mb compared with 44.4% (4 out of 9) of individuals with TMB<10 mut/Mb (HR: 3.52, 95% Cl 0.88-14.11, P=0.076) as shown in FIG. 4A.

FIG. 4B is a graphical representation of Kaplan-Meir estimates of progression-free survival for 36 individuals with MSI-H CRC stratified by progression based on response prediction model. When evaluating the predictive value of the model, progression was reported for 75.0% (3 out of 4) of non-responders compared with 18.8% (6 out of 32) of responders (HR: 6.10 95% Cl 1.45-25.69, P=0.014) as shown in FIG. 4B.

FIG. 4C is a graphical representation of the area under the curve (AUC) for prediction of ICI response for TMB and prediction model. The predictive accuracies of both TMB and the model were computed and yielded an AUC of 0.50 for TMB alone and 0.85 for the model as shown in FIG. 4C.

FIG. 4D is a graphical representation of univariate analysis to assess the predictive value of the immune response prediction model and other clinicopathological factors. The univariate analysis showed the immune response prediction model to be the only significant factor predictive of increased risk for progression (p=0.014) as shown in FIG. 4D. Survival analyses were performed using the Kaplan-Meier Estimator and the Cox method.

### Blinded Validation Cohort (BESPOKE IO)

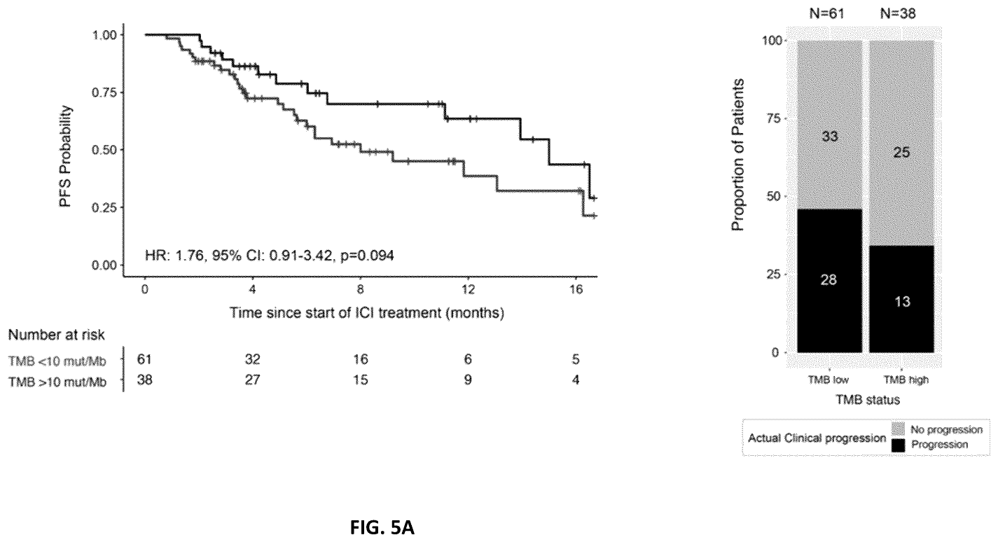

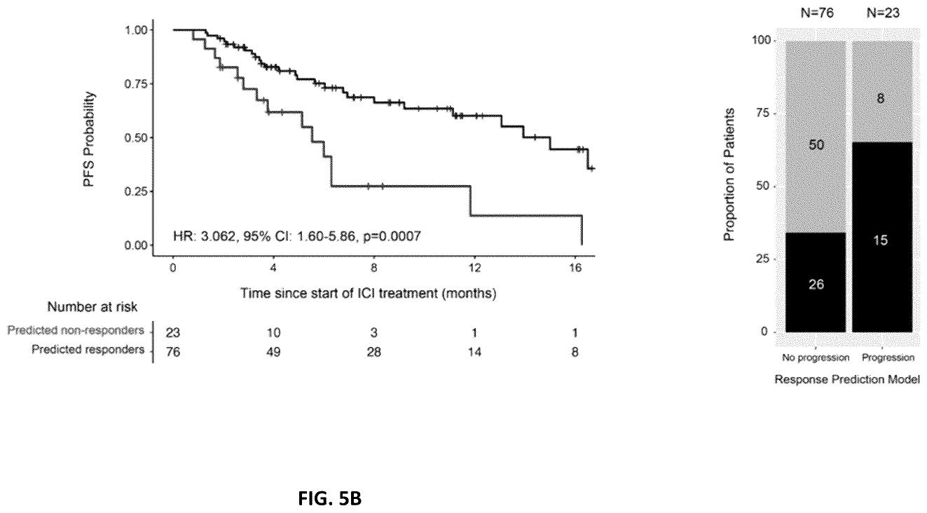

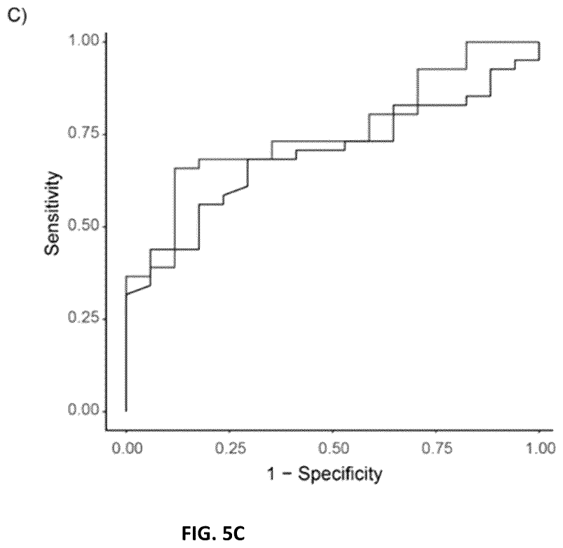

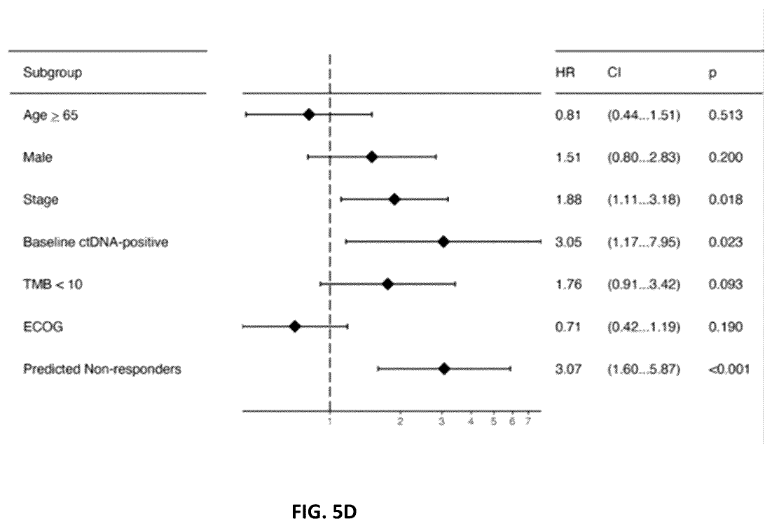

FIG. 5A is a graphical representation of Kaplan-Meir estimates of progression-free survival for 99 individuals with solid tumors stratified by TMB status. The median follow-up period for individuals in the blinded validation cohort was 10 months (range 2-35 months). When evaluating the predictive value of TMB, progression was reported for 34.2% (13 out of 38) of individuals with TMB>10 mut/Mb compared with 45.9% (28 out of 61) of individuals with TMB<10 mut/Mb (HR: 1.76, 95% Cl 0.91-3.42, P=0.094) as shown in FIG. 5A.

FIG. 5B is a graphical representation of Kaplan-Meir estimates of progression-free survival for 99 individuals with solid tumors stratified by progression based on response prediction model. When evaluating the predictive value of the model, progression was reported for 65.2% (13 out of 23) of responders compared with 34.2% (26 out of 76) of non-responders (HR: 3.06 95% Cl 1.60-5.86, P=0.0007) as shown in FIG. 5B.

FIG. 5C is a graphical representation of the area under the curve (AUC) for prediction of ICI response for TMB and prediction model. The predictive accuracies of both TMB and the model were computed and yielded an AUC of 0.70 for TMB alone and 0.75 for the model as shown in FIG. 5C.

FIG. 5D is a graphical representation of univariate analysis to assess the predictive value of the immune response prediction model and other clinicopathological factors. In univariate analysis of individual clinicopathological features for PFS, the immune response prediction model was the most significant factor predictive of increased risk for progression (p<0.001) followed by stage (p=0.018) and baseline ctDNA positivity (p=0.023). No other clinicopathologic risk factors traditionally used were significant as shown in FIG. 5D. Survival analyses were performed using the Kaplan-Meier estimator and the Cox method.

### Discussion

The immune response prediction model evaluates multiple biomarkers representing different elements known to be important for stimulating a response to ICI therapy. A benefit of the approach taken is the standard access to comprehensive genomic, clinicopathologic information, and individual outcomes to treatment through a clinical genomic database and tools to assess neoantigen burden and T-cell responses. Taken together, the immune response prediction model is able to better identify responders and non-responders to ICI therapy compared to TMB alone based on a threshold of about 10 mutations per Mb.

The immune response prediction model considers several different neoantigen characteristics that influence immunogenicity and bins them across a spectrum of scores. In this way, the results better define neoantigens and ultimately improve prediction of which individuals are most likely to respond to ICI therapy.

The immune response prediction model described incorporates incorporate both results from baseline ctDNA testing and clinicopathologic characteristics (i.e., nodal status, baseline ECOG score). The response prediction model considers both tumor-intrinsic and other peripheral features that, together, increase the predictive performance compared to using only a single data source.

RNAseq data may also be utilized. RNAseq data may improve the prediction of therapy response by retaining only neoantigen variants that are expressed with high levels of foreignness such as gene-gene fusions and aberrant splicing events.

The prediction models are developed using one or more machine learning algorithms that evaluate a comprehensive dataset and identified the optimal set of features that can accurately predict neoantigen candidates, immune stimulation using response to ICI therapy and rank neoantigen candidates. The prediction models can identify the optimal set of neoantigens that may be incorporated into personalized cancer vaccines in individuals who are most likely to benefit from ICI therapy.

### Predicting and Selecting Neoantigens

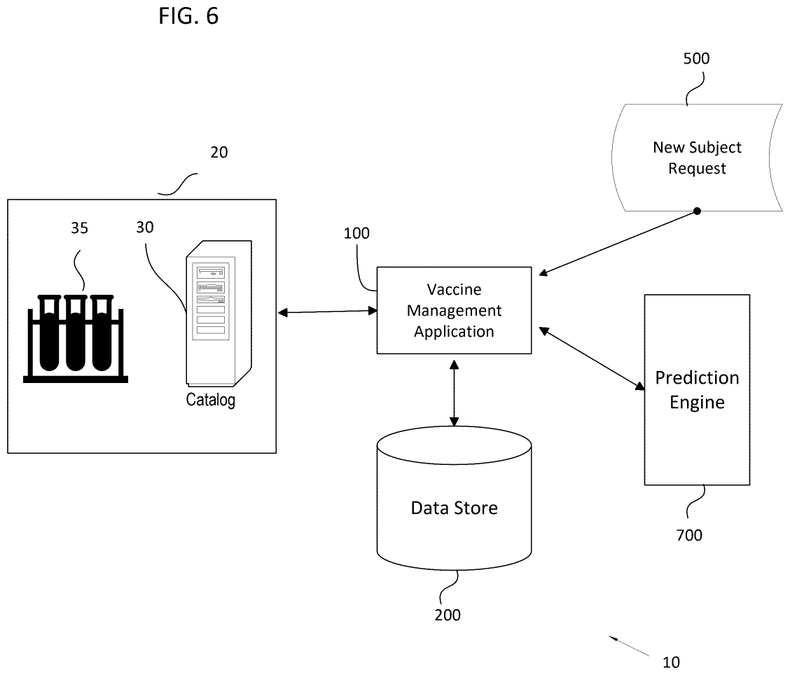

Turning now to the FIGS. 6-10, various devices, systems, and methods in accordance with aspects of the present disclosure will be described.

FIGS. 6-10 describe a system, method, and instructions for identifying and ranking the most common somatic mutations for neoantigens (candidate neoantigens) for a type of cancer and building a catalog of somatic mutations and vaccines. FIGS. 6-10 further describe a system, method and instructions for predicting immune response for an individual treated for a type of cancer.

In some examples, the common neoantigens identified represent 1-50% of addressable histology for a type of cancer. Neoantigens are peptides that arise from somatic mutations and are recognized as different from self and presented by antigen-presenting cells. The catalog of somatic mutations and vaccines for a type of cancer may then be utilized for a new subject with the type of cancer to compare DNA and RNA sequencing and determine candidate vaccines for the new subject.

Using historical data for a population of subjects treated for a type of cancer, somatic mutations for candidate neoantigens are identified for a type of cancer using population level study for somatic mutations for neoantigens. Based on the identification of common somatic mutations for a type of cancer, pools of vaccines for the type of cancer are developed using a neoantigen candidate prediction and ranking. The most clinical meaningful somatic mutations for neoantigens are determined for different cancer types. In one example, the cancer types are melanoma, lung cancer, and colorectal cancer. Using data for hundreds of subjects treated and have known outcomes for a type of cancer, the predictive model identifies somatic mutations that are candidates for vaccines. The predictive model the parameters (characteristics) of subjects and somatic mutations for neoantigens. The prediction engine identifies subjects who have responded to therapy and have tumor samples with the same or similar somatic mutation(s).

In one example, a machine learning model is used for a permutation based approach to predict neoantigen candidates, immune stimulation and rank neoantigen candidates for vaccines. Parameters are selected and used in the machine learning model to determine how subjects stratify over time after immune inhibitor therapy. Protein sequences, for neoantigens, based on DNA sequencing and RNA sequencing may be identified that that unique to a subject (or multiple subjects). The survival benefit of subjects is determined based on somatic mutations and characteristics of the resulting protein in subject samples.

It will be appreciated that any type and any combination of AI/machine learning algorithms, publicly available tools, open source tools, and commercial products may be utilized for parameter selection and determination and for the neoantigen candidate selection model, immune stimulation model and/or the neoantigen candidate ranking model (the “predictive models”) and would be expected to perform in similar ways. A person of skill would understand that various commercial models are suitable for incorporation, for example GOOGLE™ ALPHA FOLD, IntFOLD, RaptorX, HHpred, Phyre, Phyre2 and I-TASSER. Exemplary machine learning algorithms that may be utilized may include, but are not limited to, linear regression, logistic regression, decision tree, SVM algorithm, Naive Bayes algorithm, KNN algorithm, K-means, random forest algorithm, dimensionality reduction algorithms, gradient boosting algorithm, AdaBoosting algorithm, deep learning, and neural networks.

In some examples, several parameters are utilized in the machine learning model to determine which somatic mutations are vaccine candidates. The parameters include subject response to treatment such as a checkpoint inhibitor, fold change difference between a native protein for a WES germline and a protein for a somatic mutation from the tumor sample, and protein expression level of the protein for the candidate neoantigen (in some instances, using TCGA25), MHC-I: mutant peptide processing and presentation score (IQ), and the T-cell activation, dissimilarity to reference human proteome, MHC binding fold change. Additional parameters may include response to immune-checkpoint inhibitors (ICI), ctDNA results, age, sex, and ECOG score. It will be appreciated that any number of combinations of these parameters may be utilized with the neoantigen candidate model, immune stimulation model and neoantigen candidate ranking model described below.

One parameter is the expression level neoantigen for a somatic mutation. For instance, the number of who subjects have the same or similar somatic mutation and whether a is protein expressed for the somatic mutation. This may be determined based on historical data for DNA sequencing and RNA sequencing of subjects with a type of cancer. Additionally, RNA expression levels are utilized to determine whether somatic mutations are expressed as RNA and thus, may be able to produce a protein.

Another parameter is the IC50 value of subjects HLA proteins to a somatic mutation. In this example, a low IC 50 indicates a somatic mutation will strongly bind to HLA of the subject. If a protein for a somatic mutations binds to HLA better than a protein for the germline sequence, there is more likelihood the protein for the somatic mutation is shuttled to cell surface such that the immune system detects the mutated proteins.

Yet another parameter is the difference in a fold change between somatic mutation sequence and the germline sequence for a subject. A vaccine candidate may have a somatic mutation sequence that is found to bind better than the germline DNA sequence for the subject. The difference in the fold between the protein of the somatic mutation and the protein of the germline DNA sequence differentiates the somatic mutation from displaying a “self” protein on the surface of a cell. The immune system is trained to ignore “self” proteins to prevent autoimmune disorders. As such, a greater difference in folds the protein of the somatic mutation and the protein of the germline DNA sequence for a subject causes more immune stimulation when used as a vaccine. In some examples, the more difference between the proteins of somatic mutations and self-alleles, the better likelihood that immune system can detect proteins that are more different from self-proteins.

Another parameter is the difference in the sequence of the protein of a somatic mutation and the general proteome. The difference in sequence protein of the somatic mutation and the proteins of the germline DNA sequence differentiates the somatic mutation from displaying another “self” protein on the surface of a cell. As such, a greater difference between the protein of the somatic mutation and other proteins of the germline DNA may improve immune stimulation for the somatic mutation. An additional parameter is how well a T cell is stimulated to respond to a somatic mutation.

The machine learning model described herein may generate a score for each somatic mutation based on a set of parameters such as the parameters described above. These parameters may be more strongly weighted in the machine learning model than other parameters for the subjects.

In one example, the machine learning model determines most prevalent and highest scoring somatic mutation(s) for neoantigens for a type of cancer. For example, the most prevalent and highest scoring somatic mutation(s) for a type of cancer may be determined by analyzing data of 1000 subjects treated by a machine learning model.

For somatic mutations that occur in multiple subjects for a cancer type, the somatic mutations are assigned a score and rank ordered based on the set of parameters by the machine learning model to determine which somatic mutations are most likely to infer a survival benefit and/or to work as vaccines for a type of cancer. The somatic mutations that are determined to be vaccine candidates are cataloged and pre-made vaccine pool is generated for vaccine candidates.

The vaccines generated may include peptide based vaccines, mRNA vaccines or other types of vaccines. The somatic mutations for which vaccines are generated are the somatic mutations that have been determined from historical subject data to stimulate the immune system using the machine learning model. Additional adjuvants may be added to peptide based or mRNA based vaccines to improve immune system response to the vaccine including metals, bacteria, and derived toxic peptides.

Once the machine learning model is formulated, it is utilized to predict vaccines for a new subject with a type of cancer. In one example, a new subject may qualify to receive one or more pre-made vaccines based on somatic mutations for the new subject. Somatic mutations for the new subject are determined and are compared with somatic mutations cataloged by the machine learning model for the type of cancer. In some examples, the HLA genotype for the new subject is identified and it is determined if the HLA genotype is compatible with pre-made vaccines in the pool for somatic mutations. The vaccine(s) identified for the new subject and are distributed to the new subject. In some examples, a percentage of new subjects may qualify to receive a pre-made vaccine from the vaccine pool while others may not.

### Vaccine Bank

FIG. 6 is a system 10 illustrating vaccine bank 20, a vaccine management application 100, data store 200, a prediction engine 700, and a new subject data 500.

Vaccine bank 20 includes pre-made vaccines 35 stored in a housing facility comprising the suitable temperature control systems to maintain vaccines in a viable condition. Exemplary pre-made vaccines 35 may be various types of vaccines including peptide-based synthetic vaccines (epitope vaccines), messenger RNA (mRNA) vaccines, and traditional vaccines. Additional vaccine types may include gene therapy and editing such as use of adeno-associate virus (AAV) and adenomatous polyposis coli (APC) gene editing.

Vaccine bank 20 includes a vaccine catalog 30. Vaccine catalog 30 includes vaccines that have been catalogued according to predetermined characteristics for somatic mutations (neoantigens). Somatic mutations are a change in a DNA sequence of the subject between the germline sequence and the sequence of the tumor sample of the subject. The predetermined characteristics for somatic mutations may include major histocompatibility complex (MHC), genotypic information (e.g., single nucleated polymorphisms, copy number variants (CNVs), indels, gene fusions, structural variants, or a combination thereof SNPs of a specific nucleic acid sequence associated with a gene, or genomic or mitochondrial DNA.) Cataloguing may constitute creating a centralized record or database of the characteristics obtained for each somatic mutation.

The vaccine bank facilitates the selection from a plurality of samples of a pre-made vaccines suitable for administering to a subject with a type of cancer. Vaccine bank catalog may be stored on a computer or server located at or remotely from the vaccine bank 20. Vaccine catalog 30 may be communicated via any type of networking technology using any desired communication protocol and in any desired network topology.

Vaccine management application 100 is in communication with catalog 30, data store 200, and prediction engine 700. Vaccine management application 100 may include a browser based graphical user interface (GUI) or command line interface (CLI) to query catalog 30.

Prediction engine 700, described in more detail below, includes a method and system for utilizing one or more models to identify a pool or catalog of 10-100 somatic mutations for neoantigens to be used for personalized cancer vaccines. Pre-made vaccines 35 are generated for the identified somatic mutations for neoantigens and may be various types of vaccines including peptide-based synthetic vaccines (epitope vaccines), messenger RNA (mRNA) vaccines, and traditional vaccines. In some examples, for mRNA pre-made vaccines 35 generated for the identified somatic mutations, neoantigens may be banked with liquid nano particles (LNPS). In other examples, the mRNA for the pre-made vaccine and the LNP may be banked separately and combined when a new subject is identified for administration of a pre-made vaccine.

In other examples, pre-made vaccines may be banked as polymer nanostructures for peptide, mRNA or gene edit DNA vaccines. Somatic mutations for neoantigens from a genome may be banked separate from somatic mutations for neoantigens from an exome. In some examples, neoantigens and LNP are banked separately and combined when a new subject is identified for administration of a pre-made vaccine.

In one example, prediction engine 700 identifies somatic mutations (neoantigens) by identifying clusters of subjects that tend to have one or more neoantigens in common. Prediction engine 700 identifies clusters of subjects having a type of cancer that tend to have one or more neoantigens in common for example by looking at parameters for the subjects, such as at the clonal evolution of the tumor and/or using clustering algorithms. Other parameters for prediction may include copy number variation (CNV), subdomains, to detect clonality of somatic mutations in subjects.

In one example, based on the identification of somatic mutations for neoantigens by prediction engine 700, a catalog 30 and a pool of pre-made vaccines 35 are generated covering the most immunogenic somatic mutations for neoantigens in the centroid of each cluster subjects having a type of cancer.

For new subjects with a type of cancer, subjects may be matched to pre-made vaccines 35 by identifying the cluster to which the subject belongs. In one example, an immune response score is determined for each new subject for the available pre-made vaccines to match a new subject to pre-made vaccines by identifying to which cluster the patient may belongs and/or predicting an immune response score of the patient to each available pre-made vaccine. The pre-made vaccines may also be cataloged based on genome or exome DNA sequencing of the somatic mutation.

New subject data 500 may be remotely received by the vaccine management application 100. New subject data 500 may include DNA and RNA sequencing data for a germline (native DNA) and a tumor sample of a new subject. In some examples, the DNA and RNA sequencing data is whole exome sequencing (WES) germline sequencing data for a subject, WES on a tumor sample of the new subject, and RNA sequencing on the tumor sample of the new subject. In examples, new subject data is not part of historical subject data for training the predictive models but is used with the trained predictive models to predict vaccines to be administered to the new subject.

Vaccine administration to the subject(s) from the vaccine pool may be monitored and additional data may be collected. The data may include the efficacy of the vaccine for immune stimulation, treatment outcomes for the subject, additional DNA and RNA data including identification of sub clonal variants, copy number and variant allele frequency (VAR), and information regarding minimal disease resistance (MRD)

The data store 200 may be maintained by the vaccine management application 100 or may be maintained separately. A variety of other data may also be stored in the data store 200.

New subject data 500 may be received by vaccine management application 100 and utilized by prediction engine 700 with prediction models to determine or predict what vaccines to administer to the new subject.

### Prediction Engine

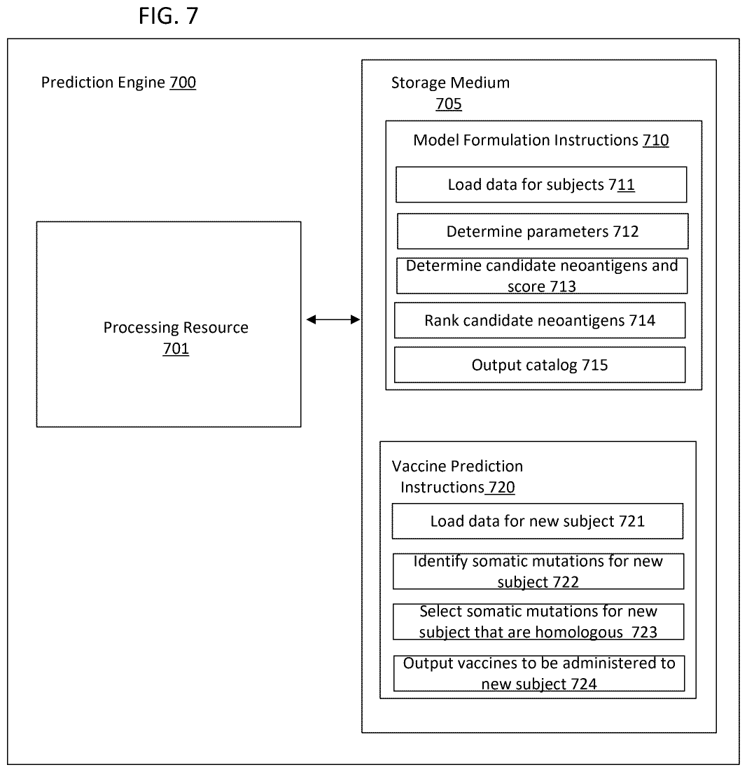

FIG. 7 illustrates one example of the prediction engine 700 in greater detail. As shown in FIG. 7, the prediction engine 700 comprises a processing resource 701 (e.g., a processor, CPU, GPU, SoC, or other processing resource), and a storage medium 705 (e.g., a non-transitory computer readable medium). The storage medium 705 comprises model training instructions 710 and model prediction instructions 720. The model training instructions 710 are executable to cause a neoantigen candidate model, immune stimulation model and neoantigen candidate ranking model to be trained, whereas model prediction instructions 720 are executable predict neoantigen candidates to be used as vaccines to be administered to a subject with a type of cancer.

Prediction engine 700 utilizes the historical data set from the data store 200 to train neoantigen candidate model, immune stimulation model and neoantigen candidate ranking model. The model training instructions 410 include instructions 711 to load historical subject data from the data store 200. The model training instructions 710 also comprise instructions 712 to determine parameters for a machine learning model.

The data for use for training the machine learning model, in this case data from the data store 200, is prepared and loaded for model development. The data may be added directly to the same directory as code for the machine learning model. The data may be uploaded as part of an experiment directory. The data may also be available using a distributed file system, to allow a cluster of machines to access the shared data set. The data may also be made available as object stores that manage data.

The parameters may include somatic mutations in subjects with a type of cancer, subject response to treatment such as a checkpoint inhibitor, fold change difference between a native protein for a WES germline and a protein for a somatic mutation from the tumor sample, and protein expression level of the protein for the candidate neoantigen (in some instances, using TCGA25), MHC-I: mutant peptide processing and presentation score (IQ), and the T-cell activation, dissimilarity to reference human proteome.

Other historical data for the subjects includes treatment used for subjects, the checkpoint inhibitor used for treatment, subject outcome data in response to treatment. Immune checkpoint inhibitors used for treatment may include a PD-1 inhibitor, a PD-L1 inhibitor, a CLTA-4 inhibitor, or a combination thereof.

In examples, prediction engine 700 compares WES sequencing data on a germline of a subject with WES sequencing data for a tumor sample from the same subject to determine changes in the DNA sequencing such as somatic mutations. Somatic mutations for neoantigens may comprise single nucleotide variants (SNV), multi-nucleotide variants (MNVs), copy number variants (CNVs), indels, gene fusions, structural variants, aberrant splice variants or a combination thereof. The sequencing data of DNA obtained from the tumor sample is compared with DNA obtained from normal subject tissue to determine somatic mutations that have occurred in the tumor sample but not the germline of the subject. Somatic mutations in the tumor sample of the patient that cause changes in protein sequences.

In some examples, the WES sequencing data from a tumor sample is compared to a reference genome to find somatic mutation and germline variants that differ from the normal reference genome. An individual may have both somatic mutations and germline variants (that are normal but differ from the reference genome.) Immune response prediction model may predict amino acid sequences that contain both types of variants. Somatic mutations are phased with germline variants to determine mutation-germline allele haplotypes. Normal germline variations, such as germline variants that are normal but differ from the normal reference genome, as well as somatic mutations are considered when making a personalized cancer vaccine for an individual.

In some examples, RNA sequencing (RNAseq) is performed and determines the presence and quantity of RNA in a biological sample, representing an aggregated snapshot of the cells' dynamic pool of RNAs, also known as transcriptome. In examples, RNA sequencing that may be utilized includes whole transcriptome sequencing, ribosomal RNA depletion, and targeted gene expression sequencing in RNA. In some examples, RNA sequencing may be inferred where RNA sequencing data is unavailable.

The predictive model may be trained in a machine learning development environment. An exemplary machine learning development environment that may be utilized to train the predictive models has an expert, one or more agents and a trial runner. The master, agents and trial runner may reside on a single computer server or may be distributed across computer servers in a cloud-computing environment.

The master is the central component and may be responsible for storing experiments, trials, and historical subject data from the data store 200. The master schedules and dispatches work to agents. The master may also be responsible for managing and deprovisioning agents in a cloud environment. The master may advance the experiment, trial, and workload state machines over time.

An agent manages a number of slots, which are computing devices, typically central processing units (CPU). An agent has no state and communicates with the master. Each agent is responsible for discovering local computing devices (slots) and sending data about them to the master. The agent runs the workloads that are requested by the master. The agent monitors containers and sends information about them to the master.

The trial runner runs a trial in a containerized environment. The trial runners are expected to have access to the data that will be used in training. The agents are responsible for reporting the states of trial runner to the master. The machine learning development environment is prepared by determining the CPU to be used for training the models. The containers may be default containers or may be customized.

The machine learning environment described above is one example of a machine learning environment in which the neoantigen candidate model, immune stimulation model and neoantigen candidate ranking model of the prediction engine 700 may be trained, but it should be understood that any other desired machine learning environment may be used.

Model training code may be converted to leverage APIs in the machine learning development environment. Hyperparameter tuning is performed to select the data, features, model architecture and learning algorithm to yield a predictive models.

Although the model training instructions 710 and model prediction instructions 720 are shown in FIG. 7 together as part of the same storage medium 705 for ease of description, in some examples these may be provided separately. For example, the prediction engine 700 may comprise multiple computing systems, including one or more systems used for training the neoantigen candidate model, immune stimulation model and one or more systems that neoantigen candidate model, immune stimulation model and neoantigen candidate ranking model.

In some examples, the system(s) used for training may have the model training instructions 710 but not the model prediction instructions 720, while conversely the system(s) used for predicting may have the model prediction instructions 720 but not the model training instructions 710. In other examples, the prediction engine 700 may comprise one or more computing systems that comprise both the model training instructions 710 and model prediction instructions 720.

### Neoantigen Candidate Selection Model

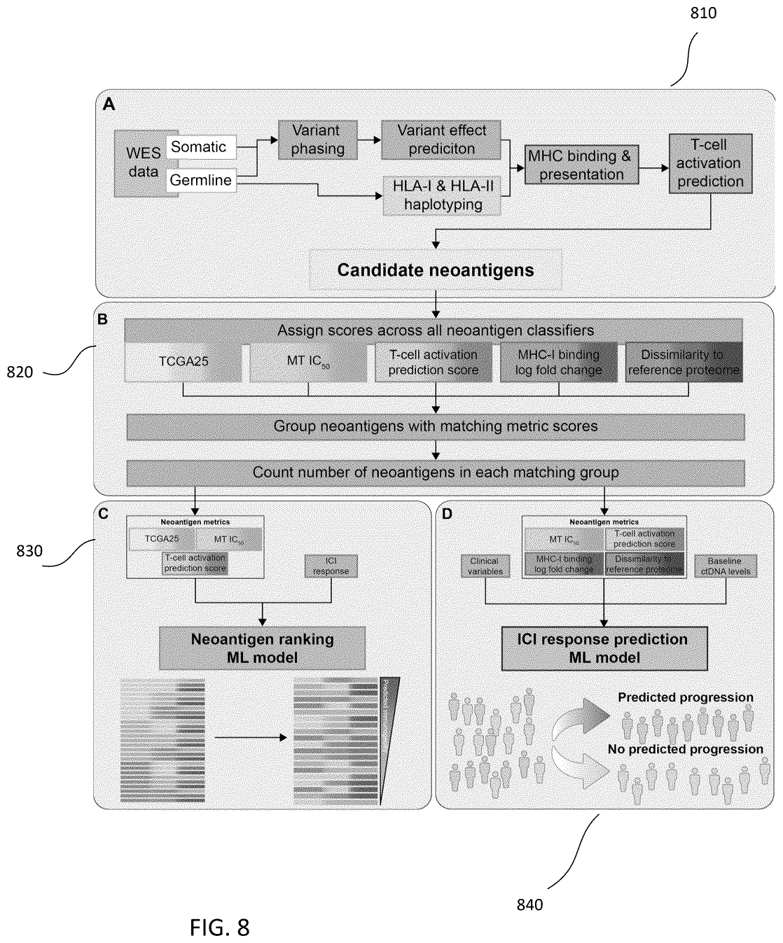

FIG. 8 depicts a bioinformatics pipeline utilizing an ensemble of standard and newly developed algorithms and models to identify and prioritize candidate immunogenic neoantigens. At 810, candidate neoantigens are identified. Tumor and matched normal WES were aligned to the reference genome and phased to determine coding sequence alterations (including both germline and somatic variants). WES data is also used for HLA-I and HLA-II haplotyping. Variants and HLA haplotypes are evaluated together to determine MHC-I and MHC-II binding and presentation. Finally, T-cell activation is predicted. While the parameters of MHC binding prediction, T-cell activation and HLA 1 haplotyping are used to predict neoantigen candidates, it will be appreciated that the parameters may be used in any combination and additional parameters may be utilized with prediction engine 700 to predict neoantigen candidates.

To identify candidate neoantigens, prediction engine 700 of FIG. 7 performs the comparison of WES sequencing data on a germline of a subject with WES sequencing data for a tumor sample from the same subject to determine changes in the DNA sequencing such as somatic mutations for a group of subjects diagnosed with a type of cancer. In embodiments, the prediction engine 700 performs the steps for each subject in the group of subjects diagnosed with the type of cancer from the sample set. the number and abundance of unique T- and B-cell receptors from RNA sequencing data for the subject, and the enrichment of immune cell populations in the tumor sample.

Prediction engine 700 determines the number of base pairs for a somatic mutation for neoantigens that differ from a normal reference sequence. In an example, prediction engine 700 identifies tumor and germline consensus single nucleotide variants (SNVs) and insertions/deletions (indels) were identified via calling algorithms using the aligned tumor and normal BAM files. Somatic SNVs and indels were phased with germline variants to determine mutation-germline allele haplotypes and then annotated (VEP v109) to determine the highest impact consequence of each mutation. MHC class-I (MHC-I) alleles were genotyped using OptiType v1.3.3. MHC class-II (MHC-II) alleles were genotyped using HISATgenotype v1.3.3.

In some examples, prediction engine 700 utilizes RNA sequencing data for the tumor sample to filter out somatic mutations in unexpressed genes. Prediction engine 700 determines RNA sequences for the somatic mutations. In one example, prediction engine 700 utilizes an in silico translation tool. The translation tool mutations translates DNA sequence changes into the protein sequences and determines the number of RNA sequences for the somatic mutation. Peptide-MHC binding prediction to determine whether the somatic mutations will bind to an individual's major histocompatibility complex (MHC) may then be conducted in two steps. First, all amino-acid sequence altering mutations in addition to MHC-I and MHC-II alleles were input into pVAC-Seq v4.0.5. pVAC-Seq is a pipeline that generates mutant and germline peptide sequences within known MHC binding lengths, produces all MHC-peptide combinations, executes many published peptide-MHC binding prediction tools, and collates the peptide-MHC binding prediction metrics. The prediction engine 700 then determines whether T-cells will bind to the MHC peptide complexes.

In examples, artificial intelligence (AI) may be utilized to assess the genetic region around somatic mutations for neoantigens to predict expression level and binding of a neoantigen. It will be appreciated that any type and any combination of AI/machine learning algorithms, publicly available tools, open source tools, and commercial products may be utilized for the parameter selection for the neoantigen candidate selection model, immune stimulation model and/or the neoantigen candidate ranking model and would be expected to perform in similar ways. A person of skill would understand that various commercial models are suitable for incorporation into the application, for example GOOGLE™ ALPHA FOLD, IntFOLD, RaptorX, HHpred, Phyre, Phyre2 and I-TASSER.

### Parameter Selection

With reference to FIG. 8, once neoantigen candidates are selected, prediction engine 700 determines additional parameters (or classifiers) for the neoantigen candidates at 820. The additional parameters include TCGA25, MT IC50 (MHC-1: mutant peptide processing and presentation score (IQ)), T-cell activation prediction score, MHC-I binding log fold change (MHC-I binding fold change log,(WT IC,0)) and dissimilarity to reference proteome. Parameters describing different aspects that have been shown to impact immunogenicity are applied and a score is assigned. Once scores are assigned, candidate neoantigens with matching characteristics are grouped together and summed.

Any suitable combination of these parameters may be used to train a machine learning model. This pipeline incorporates a combination of parameters may be utilized for prediction using the neoantigen candidate model, immune stimulation model and neoantigen candidate ranking model for vaccines. The prediction engine 700 described herein incorporates various relevant parameters, including any combination of those described above, as well as additional parameters for predicting MHC/peptide binding to determine neoantigen candidates, immune stimulation and/or candidate neoantigens to be used as vaccine candidates. Other parameters may include clonal evolution of the tumor DNA sequencing and/or using clustering algorithm parameters.

Prediction engine 700 utilizes the historical data set for a group of subjects with a type of cancer to determine characteristics or parameters of subjects that are used to train a machine learning model. In some embodiments, the group of subjects with a type of cancer have been treated for cancer.

Prediction engine 700 may infer RNA expression when RNA sequencing data is unavailable. An RNA expression database, such as The Cancer Genome Atlas (TCGA) may be utilized to infer gene expression for an individual. Prediction engine 700 determines what percentage of individuals in the RNA expression database are expressing a protein for gene for neoantigen candidate. For example, if individuals from the RNA expression database express the particular gene>=25th percentile, RNA expression may be inferred. Prediction engine 700 may also permute through neo-antigen properties using an RNA expression database. For example, it may determine the proportion of individuals with cancer expressing a gene, such as a somatic mutation. Determining whether RNA is expressed Exemplary parameters may include the expression level of a somatic mutation. This may be determined based on data for DNA sequencing and RNA sequencing of subjects with a type of cancer. Additionally, RNA expression levels are utilized to determine whether somatic mutations are expressed as RNA and thus, may be able to produce a protein. In examples, commercially available Al tools may be utilized predict RNA expression level using DNA and genetic motif data.

Prediction engine 700 also determines the MT IC50 (MHC-1: mutant peptide processing and presentation score (IQ)), for neoantigen candidates to predict those that strongly bind to the individual's MHC alleles and/or are immunogenic (e.g., IC₅₀<600 nM, or IC₅₀<550 nM, or IC₅₀<500 nM, or IC₅₀<450 nM, or IC₅₀<400 nM; and/or percentile rank<0.6%, or percentile rank<0.55%, or percentile rank<0.5%, or percentile rank<0.45%, or percentile rank<0.4%).

The IC50 value of subjects HLA proteins to a somatic mutation sequence may be utilized. In this example, a low IC50 indicates a somatic mutation will strongly bind to HLA of the subject. If protein for a somatic mutations binds to HLA better than a protein for the germline sequence, there is more likelihood the protein for the somatic mutation is shuttled to cell surface such that the immune system detects the mutated proteins.

Prediction engine 700 determines the ratio of binding of a mutant peptide compared to the binding of the wild type peptide (Log2 WT IC50/MT IC50). Prediction engine 700 prefers stronger mutant peptide binding to MHC than wild type peptide binding.

Prediction engine 700 determines a T-cell activation score for neoantigen candidates. The T-cell activation score may be determined by quantifying the number of unique T- and B-cell receptors comprising (i) deconvoluting proportions of immune cells in the tumor sample based on RNA sequencing data, and (ii) assembling B and T-cell receptors to quantify the number of unique T- and B-cell receptors. T-cell activation score may be a percent rank and RNA expression levels.

Yet another parameter is the difference in a fold change between somatic mutation sequence and the germline sequence for a subject. A vaccine candidate may have a somatic mutation sequence that is found to bind better than the germline DNA sequence for the subject. The difference in the fold between the protein of the somatic mutation and the protein of the germline DNA sequence differentiates the somatic mutation from displaying a “self” protein on the surface of a cell. The immune system is trained to ignore “self” proteins to prevent autoimmune disorders. As such, a greater difference in folds the protein of the somatic mutation and the protein of the germline DNA sequence for a subject causes more immune stimulation when used as a vaccine. In some examples, the more difference between the proteins of somatic mutations and self-alleles, the better likelihood that immune system can detect proteins that are more different from self-proteins.

Another parameter is the difference in the sequence of the protein of a somatic mutation and the general proteome. The difference in sequence protein of the somatic mutation and the proteins of the germline DNA sequence differentiates the somatic mutation from displaying another “self” protein on the surface of a cell. The immune system is trained to ignore “self” proteins to prevent autoimmune disorders. As such, a greater difference between the protein of the somatic mutation and other proteins of the germline DNA may improve immune stimulation for the somatic mutation.

A random forest algorithm may be used for parameter selection and model training. While a random forest algorithm was utilized in examples, it will be appreciated that any type and any combination of AI/machine learning algorithms, publicly available tools, open source tools, and commercial products may be utilized for the parameter selection for the neoantigen candidate selection model, immune stimulation model and/or the neoantigen candidate ranking model and would be expected to perform in similar ways. A person of skill would understand that various commercial models are suitable for incorporation into the application, for example GOOGLE™ ALPHA FOLD, IntFOLD, RaptorX, HHpred, Phyre, Phyre2 and I-TASSER.

### Immune Response Model

With reference to FIG. 8, at 830, prediction engine 700 develops a model to predict which patients are more likely to progress or not progress following ICI therapy. The model incorporates various clinical parameters, including baseline ctDNA results, and candidate neoantigens based on mutant IC50, MHC-I binding fold change, dissimilarity of the candidate neoantigen to the reference human proteome, and the T-cell activation prediction score.

A machine learning tool is provided to predict individual response to immune checkpoint inhibitor (ICI) treatment based on a large cohort of individuals with complete clinical information. By utilizing whole exome sequencing data, ctDNA time point analyses, and clinical data for parameters, a deep learning immune response prediction model successfully predicts which individuals who respond to and benefit from ICI treatment across multiple cancer types. Individuals who respond to ICI treatment may have multiple neoantigens or strong neoantigens.

In the example illustrated in FIG. 7, the immune stimulation model comprises a machine learning random survival forest algorithm. A random forest algorithm is a nonparametric machine learning strategy that can be used for building a risk prediction model in survival analysis. In survival settings, the predictor is an ensemble formed by combining the results of many survival trees. While a random forest algorithm was utilized in examples, it will be appreciated that any suitable type and combination of machine learning algorithms may be utilized for the immune stimulation model.

While a random forest algorithm was utilized in examples, it will be appreciated that any type and any combination of Al/machine learning algorithms, publicly available tools, open source tools, and commercial products may be utilized for the parameter selection for the neoantigen candidate selection model, immune stimulation model and/or the neoantigen candidate ranking model and would be expected to perform in similar ways.

### Neoantigen Ranking Model

With reference to FIG. 8, at step 840, candidate neoantigens are ranked by a machine learning model based on ICI response, TCGA25, mutant IC50, and the T-cell activation prediction score. The ICI response in a training set was evaluated in the context of neoantigens with matching sets of scores from these three characteristics. In some embodiments, ICI response may be determined using the immune stimulation model described above.

Candidate neoantigens are assigned weighted scores based on associations between neoantigens and ICI response, and subsequently used to rank neoantigens. Prediction engine 700 assigns somatic mutations for neoantigens (candidate neoantigens) a score and rank orders them based on the set of parameters, as described above, using the machine learning model to determine which somatic mutations are most likely to infer a survival benefit and/or to work as vaccines for a type of cancer. The somatic mutations that are determined to be vaccine candidates are cataloged and pre-made vaccine pool is generated for vaccine candidates.

One or more parameters may be utilized to predict candidates neoantigens for use as vaccine candidates. Any combination of the parameters described herein may be utilized to predict and rank candidate neoantigens.

The model training instructions 710 include instructions 713 to determine a score to predict the probability of certain classes (neoantigen candidates, immune stimulation and neoantigen ranking for vaccine candidacy) based on a combination of parameters. In an example, random survival forest can determine a score for neoantigens for vaccine candidacy. The somatic mutations with the greatest probably of being immune stimulating and candidates for vaccines 714. While a random forest algorithm was utilized in examples, it will be appreciated that any type and any combination of Al/machine learning algorithms, publicly available tools, open source tools, and commercial products may be utilized for the parameter selection for the neoantigen candidate selection model, immune stimulation model and/or the neoantigen candidate ranking model and would be expected to perform in similar ways. A person of skill would understand that various commercial models are suitable for incorporation into the application, for example GOOGLE™ ALPHA FOLD, IntFOLD, RaptorX, HHpred, Phyre, Phyre2 and I-TASSER.

### Vaccine Catalog

The model training instructions 710 include instructions 715 to output a catalog the somatic mutations determined as being immune stimulating and candidates for vaccines. For example, the somatic mutations that occur in multiple subjects with a type of cancer and are ranked high by the machine learning model are cataloged as being immune stimulating and a candidate for a vaccine. The catalog may include DNA sequencing and RNA sequencing data, parameters used for selection of the somatic mutation as well as scoring and ranking information.

In some examples, some of the data from the data store 200 may be used for training and other data may be used for testing and validation. In one example, 80% of the data set used from the data store 200 may be used for training the gradient boosting regression algorithm and the remaining 20% may be used for immune stimulation and vaccine candidate predictions from the predictive model.

In some examples, a user may determine when the model is sufficiently trained based on the tested error or deviance, for example with the aid of a plot of test set deviance. In other examples, the prediction engine 700 may be configured with logic to identify when the model is sufficiently trained. For example, when the test error rate is equal to or lower than a threshold (or equal to or lower than the threshold consistently for a defined number of test runs, to account for variances in the error), the prediction engine 700 may identify the model as being sufficiently trained.

In some examples, a user may designate which features (parameters) should be removed, for example with the aid of the aforementioned plot of feature importance. In other examples, the prediction engine 700 may be configured with logic to select which features to remove. For example, all features that have a relevance less than a defined relevance threshold may be omitted.

### Vaccine Prediction for New Subject

Referring again to FIG. 7, when new subject data is received for a type of cancer, immune stimulation and vaccine candidates are predicted using the prediction engine 700.

In one example, the vaccine management application 100 makes a representational state transfer (REST) application programming interface (API) call to the prediction engine 700. The payload of the REST API call includes data for the immune stimulation and vaccine prediction for each new subject.

In an example, in response to the API call, the model prediction instructions 720 are executed. The model prediction instructions 720 comprise instructions 721 to load new subject data. New subject data 500 may include DNA and RNA sequencing data for a germline (native DNA) and a tumor sample of a new subject. In some examples, the DNA and RNA sequencing data is whole exome sequencing (WES) germline sequencing data for a subject, WES on a tumor sample of the new subject, and RNA sequencing on the tumor sample of the new subject. In examples, new subject data is not part of historical subject data for training predictive models but is used with the trained predictive models to predict immune stimulation and candidate vaccines to be administered to the new subject.

The model prediction instructions 720 further comprise instructions 722 to identify somatic mutations for the new subject by comparing data for the WES germline with data for the WES tumor sample for the new subject. Model prediction instructions 720 further comprise instructions 723 for selecting somatic mutations for new subject that are homologous to somatic mutations cataloged by the machine learning model. In some examples, the somatic mutation of a new subject is 70%, 80%, 90%, or 99% homologous to one or more somatic mutations cataloged by the machine learning model.

In one example, based on the identification of somatic mutations (neoantigens) by prediction engine 700, a catalog 30 and a pool of pre-made vaccines 35 are generated covering the most immunogenic somatic mutations (neoantigens) in the centroid of each cluster subjects having a type of cancer.

For new subjects with a type of cancer, subjects may be matched to pre-made vaccines 35 by identifying the cluster to which the subject belongs. In one example, an immune response score is determined for each new subject for the available pre-made vaccines to match a new subject to pre-made vaccines by identifying to which cluster the patient may belongs and/or predicting an immune response score of the patient to each available pre-made vaccine. Instructions 424 output predicted immune stimulation and vaccine candidates for the one or more somatic mutations for the new subject. The somatic mutations, DNA sequencing and RNA sequencing for the new subject are input into the trained model to determine predicted immune stimulation and vaccine candidates for the new subject. In other words, the new subject data from the payload of the REST API is loaded into the trained predictive models to generate predicted immune stimulation and vaccine candidates.

### Methods

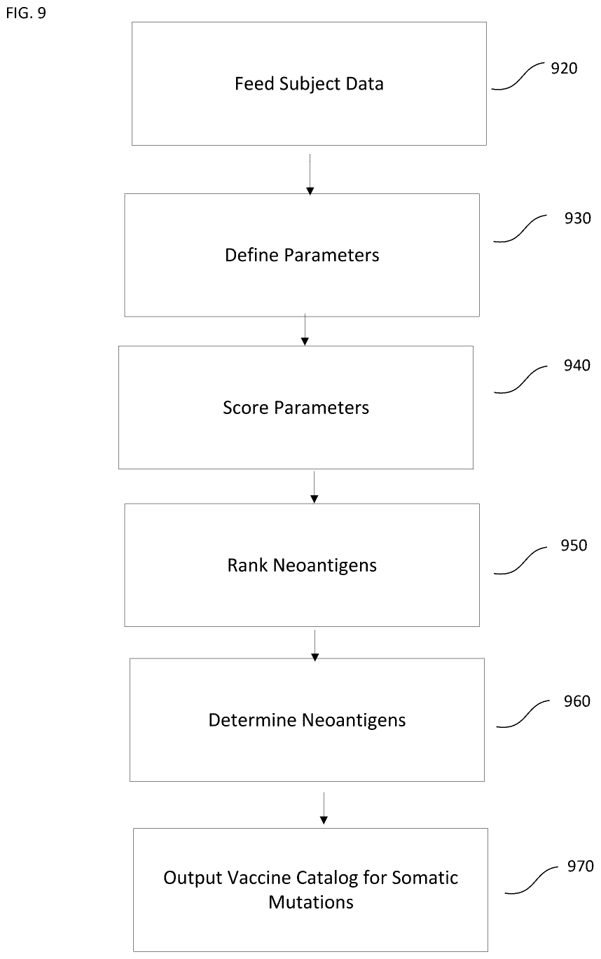

FIG. 9 is a flow diagram illustrating a method for training an immune stimulation prediction model in accordance with examples set forth herein. The method may be performed by any suitable processor or other hardware discussed herein, for example, a processor or hardware included in prediction engine 700. In particular, in some examples, the prediction engine 700 or components thereof are instantiated by one or more processors executing machine readable instructions that comprise, at least in part, instructions corresponding to the operations of the method of FIG. 9.

The method starts at step 920 when subject data from the data store 200 is loaded or fed for use by prediction engine 700. The data comprises historical data for subjects treated for a type of cancer.

At step 930, the prediction engine 700 defines the parameters for the machine learning model. In some examples, feature importance is plotted by prediction engine 700. The prediction engine 700 determines which of the subject parameters (features) satisfy a threshold of relevance and are predictive features for neoantigen candidate selection, immune stimulation and neoantigen ranking for vaccine candidacy based on determined mathematical relationships. The prediction engine 700 determines mathematical relationships between parameters and neoantigens by feeding historical subject data as inputs into one or more machine learning models.

At step 940, a score is determined for each of the neoantigens for at least some the subjects, by using the data from subjects previously treated for a type of cancer. The scores for each of the immune stimulation parameters for at least some of the subjects are aggregated and an aggregated score is generated for the candidate neoantigens that occur in a subset of the subjects.

At step 950, each of the neoantigens that occur in the subset of the subjects are ranked by the aggregated scores. Based on the predicted probability, the somatic mutations are ranked. The somatic mutations with the greatest probability of being immune stimulating and candidates for vaccines are ranked the highest.

At step 960, the predicted somatic mutations for the neoantigens is determined. The somatic mutations for the neoantigens may be determined based on data for the subjects treated for a type of cancer is processed to determine somatic mutations for the subjects by comparing tumor DNA sequencing to germline DNA sequencing for a subject. The subject data may also be preprocessed by formatting for the prediction engine, checking for completeness and bias, checking for and imputing missing values, and smoothing and binning the data.

At step 970, a catalog is output for somatic mutations for neoantigens that are immune stimulating and are candidates for vaccines. For example, the somatic mutations for neoantigens that occur in multiple subjects with a type of cancer and are ranked high by the machine learning model are cataloged as being immune stimulating and a candidate for a vaccine. The catalog may include DNA sequencing and RNA sequencing data, parameters used for selection of the somatic mutations for neoantigens as well as scoring and ranking information.

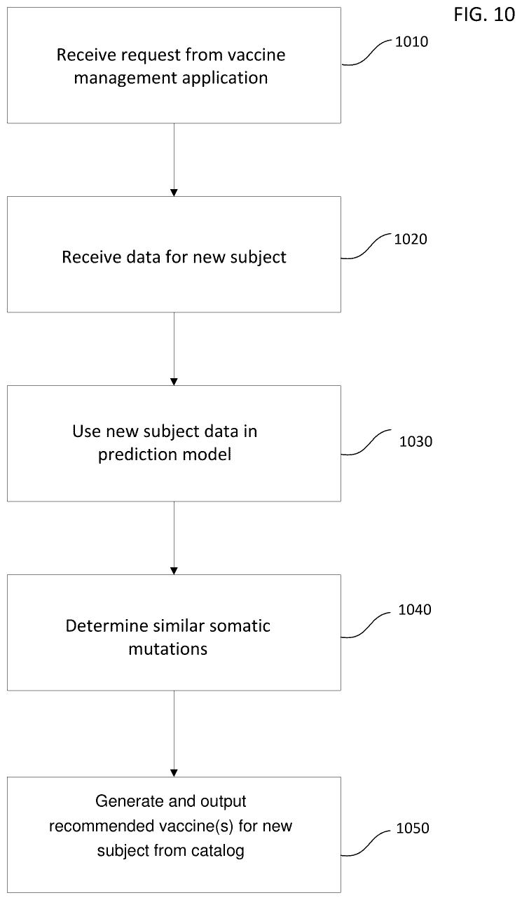

Utilizing the steps of FIG. 9, a trained model is generated. The trained model can be utilized for predicting immune stimulation and vaccine candidates for a new subject as described in FIG. 10.

FIG. 10 is a flow diagram illustrating a method for predicting immune stimulation and vaccine candidates for a new subject in accordance with examples set forth herein. The method may be performed by any suitable processor or other hardware discussed herein, for example, a processor or hardware included in prediction engine 700. In particular, in some examples, the prediction engine 700 or components thereof are instantiated by one or more processors executing machine readable instructions that comprise, at least in part, instructions corresponding to the operations of the method of FIG. 10.

A method starts in step 1010, in which the prediction engine 700 receives a request from a vaccine management application. At step 1020, data for a new subject is received. New subject data 500 may include DNA and RNA sequencing data for a germline (native DNA) and a tumor sample of a new subject. In examples, the DNA and RNA sequencing data is whole exome sequencing (WES) germline sequencing data for a subject, whole genome sequencing, panels of cancer genes, WES on a tumor sample of the new subject, and RNA sequencing on the tumor sample of the new subject.

In step 1030, the prediction engine 700 determines predicted immune stimulation and vaccine candidates for one or more somatic mutations for the new subject. The new subject data is fed as inputs into the immune stimulation prediction model generated by prediction engine 700.

In step 1040, the prediction engine 700 determines from a catalog of somatic mutations for subjects treated for a type of cancer that are homologous to one or more somatic mutations for the new subject.

In step 1050, the prediction engine 700 generates and formats predicted immune stimulation and vaccine candidates for one or more somatic mutations for the new subject. In one example, the predicted immune stimulation and vaccine candidates are returned as payload data. Vaccine candidates may then be selected from vaccine bank having a pool of pre-made vaccines for somatic mutations for different types of cancer.

### Example 2

In another example, patients responding well to current immunotherapies are enriched for immunogenic neoantigens and patient responses to ICIs inform better neoantigen prediction and prioritization. Also, these neoantigen characteristics help predict patient responses to ICIs.

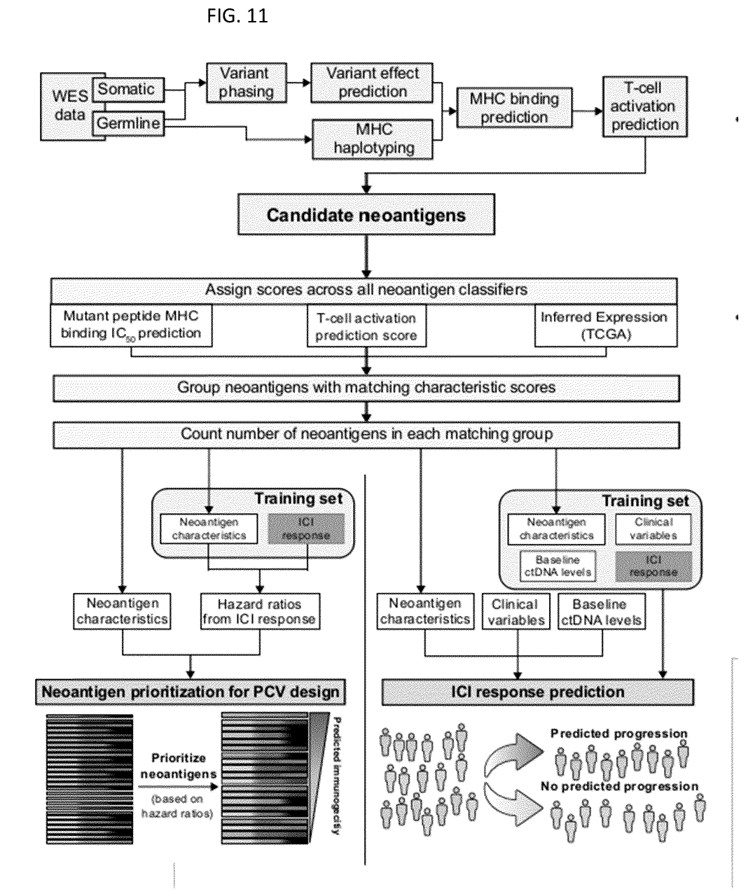

As shown in FIG. 11, candidate neoantigen selection and prioritization for PCV design and ICI response prediction and prioritization was performed. Tumor and matched normal WES was data used to identify candidate neoantigens. Candidate neoantigens binned according to MHC-I binding, predicted T-cell immunogenicity, and gene expression (based on TCGA). ICI response measured by progression-free survival (from real-world clinical cohort). A strong correlation was observed between predicted neoantigen immunogenicity and ICI outcomes using Cox proportional hazard ratios.

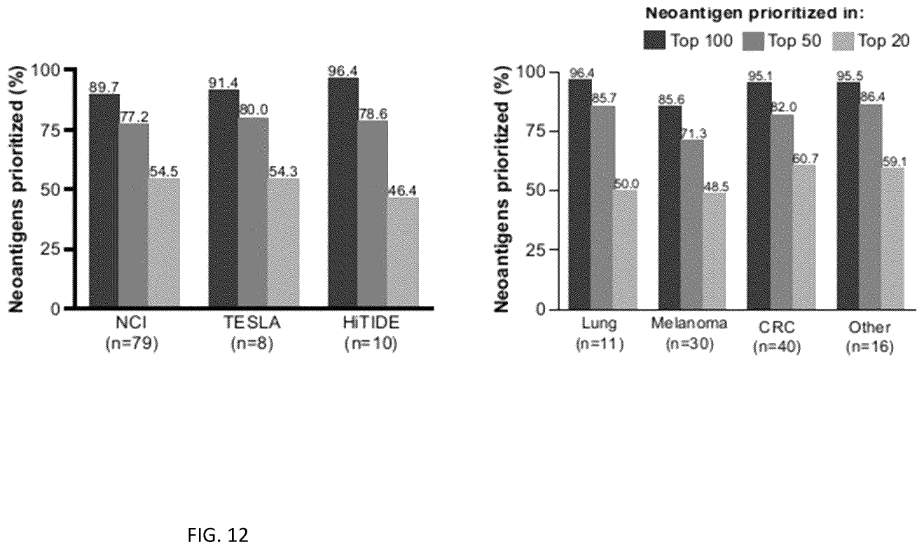

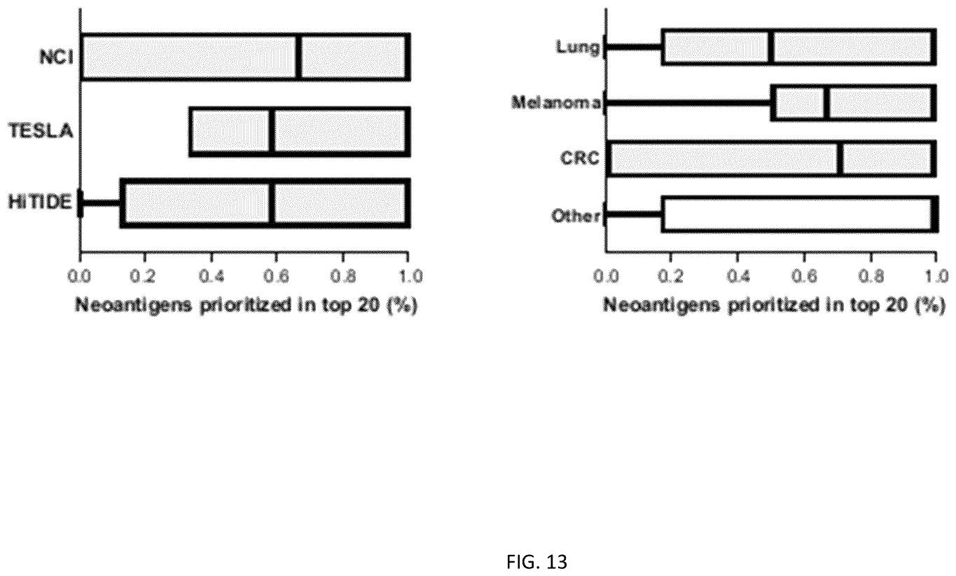

As shown in FIG. 12, prioritization performance neoantigens using the neoantigen prediction method was assessed using publicly available benchmarking datasets. NCI1, TESLA2, and HiTIDE3 datasets were used with known immunogenic neoantigens as assessed by in vitro experiments. Approximately half of true-positive neoantigens were identified by the neoantigen prediction and prioritization method when prioritizing the top 20 neoantigens, a common number of neoantigen targets included in current PCVs. FIG. 13 is boxplots for each patient, depicting the proportion of true-positive neoantigens prioritized in the top 20 by the neoantigen prediction and prioritization method.

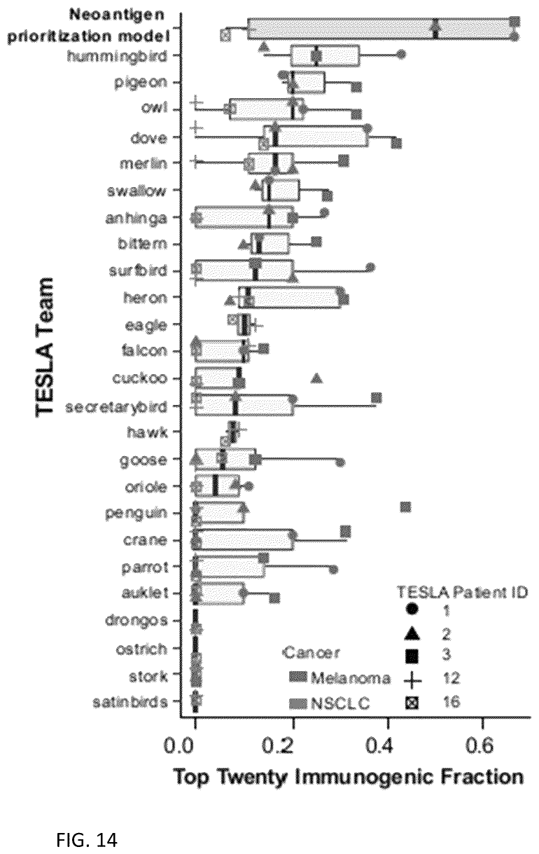

As shown in FIG. 14, the neoantigen prediction and prioritization method described herein performs better than other prediction models. The results of the neoantigen prediction and prioritization method, described herein, were compared to other neoantigen prioritization methods in the TESLA cohort including five patients (three melanoma, two NSCLC).

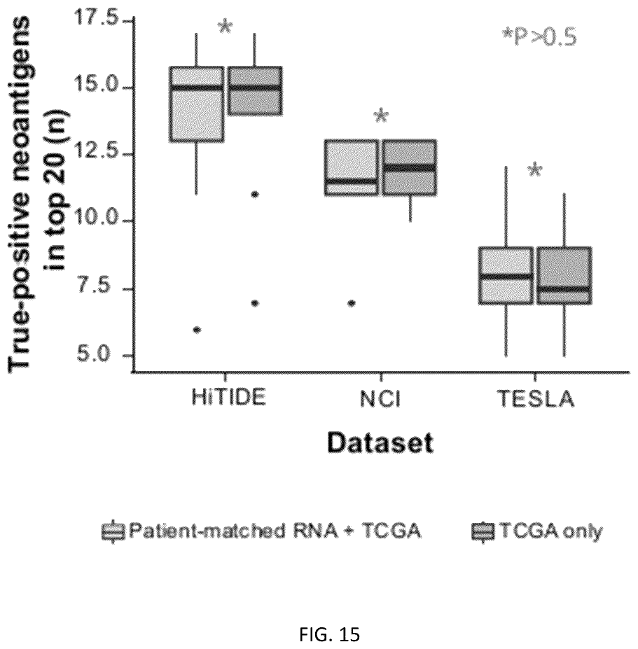

Referring next to FIG. 15, in this example, the Inclusion of patient-matched RNAseq data had little impact on neoantigen prioritization performance. FIG. 15 depicts the number of true-positive neoantigens selected by neoantigen prediction and prioritization models trained with and without patient-matched RNA. In some instances, population-based data from the TCGA is sufficient for gene expression inference by the neoantigen prediction and prioritization model. Although in other examples, RNA data may in enhance the neoantigen and prioritization method.

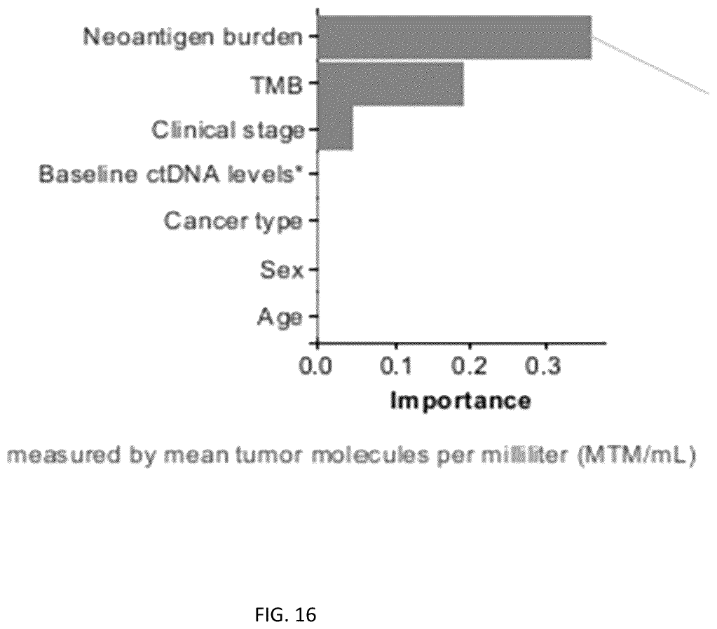

As shown in FIG. 16, the neoantigen prediction and prioritization method provides better ICI response prediction than using TMB alone. In this example, neoantigen burden was the most predictive factor in classifying patients as responders and non-responders. Kaplan-Meier estimates of PFS shows that the neoantigen prediction and prioritization method classifies patients more accurately into responders and non-responders than using TMB.

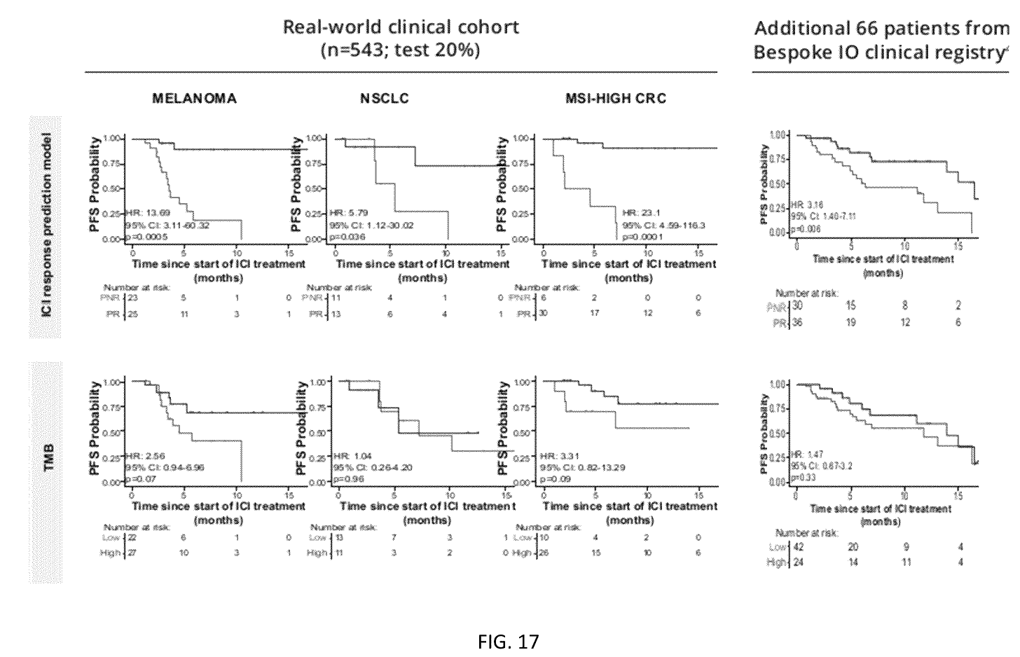

As shown in FIG. 17, the neoantigen prediction and prioritization method is informed by in vivo ICI response. The neoantigen prediction and prioritization method effectively identifies immunogenic targets for PCV design and can be used to avoid ICI treatment in patients unlikely to benefit.

The methods, systems, devices, and equipment described herein may be implemented with, contain, or be executed by one or more computer systems. The methods described above may also be stored on a non-transitory computer-readable medium. Many of the elements may be, comprise, or include computers systems.

It is to be understood that both the general description and the detailed description provide examples that are explanatory in nature and are intended to provide an understanding of the present disclosure without limiting the scope of the present disclosure. Various mechanical, compositional, structural, electronic, and operational changes may be made without departing from the scope of this description and the claims. In some instances, well-known circuits, structures, and techniques have not been shown or described in detail in order not to obscure the examples. Like numbers in two or more figures represent the same or similar elements.

In addition, the singular forms “a”, “an”, and “the” are intended to include the plural forms as well, unless the context indicates otherwise. Moreover, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electronically or mechanically directly coupled, or they may be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one example may, whenever practical, be included in other examples in which they are not specifically shown or described. For example, if an element is described in detail with reference to one example and is not described with reference to a second example, the element may nevertheless be claimed as included in the second example.

Further modifications and alternative examples will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various examples shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the scope of the present teachings and following claims.

It is to be understood that the particular examples set forth herein are non-limiting, and modifications to structure, dimensions, materials, and methodologies may be made without departing from the scope of the present teachings.

Other examples in accordance with the present disclosure will be apparent to those skilled in the art from consideration of the specification and practice of the disclosure herein. It is intended that the specification and examples be considered as exemplary only, with the following claims being entitled to their fullest breadth, including equivalents, under the applicable law.

1. A system comprising:
at least one computing device comprising at least one processor configured to: feed data from subjects into a predictive model;
score neoantigens that occur in data from a subset of the subjects for one or parameters;
determine, based on scores for the one or more parameters, one or more neoantigens that occur in a subset of the subjects that satisfy an immune stimulation threshold;
determine somatic mutations for the one or more neoantigens that satisfy the immune stimulation threshold that occur in the subset of the subjects; and
output a predicted catalog of one or more of the somatic mutations that occur the subset of the subjects that satisfy the immune stimulation threshold predict the somatic mutations.

2. The system of claim 1, wherein data is from subjects previously treated for the type of cancer.

3. The system of claim 1, wherein the predictive model is more predictive of immune stimulation for the subset of subjects than tumor mutational burden (TMB) status.

4. The system of claim 2, wherein the predictive model is a machine learning model.

5. The system of claim 4, wherein the one or more parameters comprise a combination of one or more of peptide processing and presentation, RNA expression, MHC binding fold change, T-cell activation, and dissimilarity from reference human proteome.

6. The system of claim 5, wherein the parameters further comprise one or more of immune checkpoint inhibitor (ICI), response, ctDNA results, age, sex, and ECOG score.

7. A system comprising:
at least one computing device comprising at least one processor configured to:
feed data for a newly diagnosed subject with a type of cancer into a predictive model;
score neoantigens that occur in data for the subject for one or more parameters;
determine, based on scores for the one or more parameters, one or more neoantigens that occur for the subject, that satisfy an immune stimulation threshold;
determine somatic mutations for the one or more neoantigens that satisfy the immune stimulation threshold that occur for the subject; and
output one or more vaccines to be administered to the subject for the somatic mutations for the one or more neoantigens that satisfy the immune stimulation threshold where the one or more vaccines to be administered to the subject are selected from a predicted catalog of somatic mutations that occur in a subset of subjects previously treated for the type of cancer.

8. The system of claim 7, wherein the one or more vaccines are selected from a pool of pre-made vaccines to be administered.

9. The system of claim 8, wherein the vaccine is one or more of a peptide-based synthetic vaccine, messenger RNA (mRNA) vaccines, or traditional vaccine.

10. A non-transitory computer-readable medium storing instructions executable by a processor to cause the processor to:
at least one computing device comprising at least one processor configured to: feed data from subjects into a predictive model;
score neoantigens that occur in data from a subset of the subjects for one or more parameters;
determine, based on scores for the one or more parameters, one or more neoantigens that occur in a subset of the subjects that satisfy an immune stimulation threshold;
determine somatic mutations for the one or more neoantigens that satisfy the immune stimulation threshold that occur in the subset of the subjects; and
output a predicted catalog of one or more of the somatic mutations that occur the subset of the subjects that satisfy the immune stimulation threshold predict the somatic mutations.

11. The non-transitory computer-readable medium of claim 10, wherein data is from subjects previously treated for the type of cancer.

12. The non-transitory computer-readable medium of claim 10, wherein the predictive model is more predictive of immune stimulation for the subset of subjects than tumor mutational burden (TMB) status.

13. The non-transitory computer-readable medium of claim 11, wherein the predictive model is a machine learning model.

14. The non-transitory computer-readable medium of claim 13, wherein the parameters comprise a combination of one or more of peptide processing and presentation, RNA expression, MHC binding fold change, T-cell activation, and dissimilarity from reference human proteome.

15. The non-transitory computer-readable medium of claim 14, wherein the one or more parameters further comprise one or more of immune checkpoint inhibitors (ICI) response, ctDNA results, age, sex, and ECOG score.

16. A vaccine composition, prepared by a process comprising the steps of:
feeding data for a subject with a type of cancer into a predictive model;
scoring neoantigens that occur in data for the subject for one or more parameters;
determining, based on scores for the one or more parameters, one or more neoantigens that occur for the subject, that satisfy an immune stimulation threshold;
determining somatic mutations for the one or more neoantigens that satisfy the immune stimulation threshold that occur for the subject; and
preparing one or more vaccine compositions to be administered to the subject for one or more somatic mutations for one or more neoantigens that satisfy an immune stimulation threshold where the one or more vaccines to be administered to the subject are selected from a predicted catalog of somatic mutations that occur in a subset of subjects previously treated for the type of cancer.

17. The vaccine composition of claim 16, wherein the one or more parameters comprise a combination of one or more of peptide processing and presentation, RNA expression, MHC binding fold change, T-cell activation, and dissimilarity from reference human proteome.

18. The vaccine composition of claim 16, wherein the one or more vaccine compositions are selected from a pool of pre-made vaccine compositions to be administered.

19. The vaccine composition of claim 16, wherein the one or more vaccine compositions is one or more of a peptide-based synthetic vaccine, messenger RNA (mRNA) vaccines, or traditional vaccine.

20. The vaccine composition of claim 19, wherein process further comprises:
combining the one or more neoantigens and liquid nanoparticle (LNP) to prepare the one or more vaccine compositions.
